SEQUENCE LISTING (14 sequences)

SEQ ID NO: 1 - DNA, 222 bp, Homo sapiens
gtgtcgctgctgtgggaggcggcggcagagtgaggggcgaggcccgaggggccggcggcggcggcggcggcggcggcggcggcggcggcggcggcggcggcgggggcttcccggccgggtgagcggctcggggggaggcggggcgaggaggcctgggcccgcagggagggccgtgtccgccacccaggaggggcggttctcggggtggcggatggggcagcg

SEQ ID NO: 2 - DNA, 24 bp, Artificial Sequence
  Description of Artificial Sequence Primer
gtgggaggcggcggcagagtgagg

SEQ ID NO: 3 - DNA, 24 bp, Artificial Sequence
  Description of Artificial Sequence Primer
gccccatccgccaccccgagaacc

SEQ ID NO: 4 - DNA, 6882 bp, Homo sapiens
  misc_feature (500): a, t, c, g, unknown or other
gccgggcatggtggcgcacacctgtaatcccagctactcaggaggctgaggcaggaggatcacttgaacctgggaggcagaggttgcagtgagccgagatcgcaccactgcactccagcctgggtgacaagagccagactctatctctaaataaataaataggccgtgcgctgtggctcacgcctgtaatctcagcactctgggaggctgaggcgggaggatcacctgaggtcaggagttcgagaccagcctggccgacatggggaaaccctgtctctactaaaaaaaaaaaaaaaattagccgggcatggtggcgcagcctgtaatcccagctactcaggaggctgaggcaggagaatcacttgaacccgggaggcagaggttgcagtgaaccgagatcacaccactgcactccagcatgggtgacagagattctgtctcaaaataaataaataagtaaaaataaaaaagtggttgtagtcaaaggctatgggtttaangttgggtagtgggatttcaacatttaaggttttagagatggagcagtatcaagtgatgaatatctagattttctagactacgggtgaacaaagtagaagtaaaggtcattcggattgaggaactgaaggaaaccaggctgggttgtgsatgttggaattcattgggagcgatggcatgtaaatcagatggagaggaaaactaatgccagacagtcagtaggtgacagtgacaagggagggcaaaaggtgttactaccagatggcacagagcctgaaagagctatttcacaaggggaagaaagaatatcagtgtcaagcggaaagcttcaagagtgaagaagacacactacctttgaggctggtgcatggagtgtgggaggggagaaaccaagcgagtagacagagatgaggcaatcttttgaattggatgacggatagatgttggtaccatccgaagcatttgggaagtccagaggggaggctttgttttccgggaatatgcttaatttgaggagtgggaaatggatagagggtaggtgaatggttagagatctgcacaaagctggaaggcccacgggatcaagttctgcccatgactgaggggtgacctccaatccctgggccttaacccccaggatccctggacctggaacgcccattaggtcccctcctttccggcgagagggcggcagagggcgcaggcgctctagccggacccggcgggcgggtggcagagggcgcaggcgccctggacagagcggccgcagtgggaacggccggcagggggcgcagcgctctggtccgagaagcccgggcggggcaccctcgccgggagagtagaggataggggtcggctggagaacgggcccagtgcgaggccgcgagcagtgggcggggccggggtgggggcmggggcgaggcggttccggaaggaagtggttccgggtcccgcggcccggcagaggtggctggggtgtcgctgctgtgggaggcggcggcagagtgaggggcgaggcccgaggggccggcggcggcggcggcggcggcggcggcggcggcggcggcggcgggggcttcccggccgggtgagcggctcggggggaggcggggcgaggaggcctgggcccgcagggagggcgtgtccgccacccaggaggggcggttctcggggtggcggatggggcagcggggaggatgcggcggtccgactcgggttctgttgggacagctcctctgcgccccgtaggtccctcggtcagcgccttcccttgggcgcggcgacccggcgcgggtttccaggggacagctgcccggcgcccctgaggtccctcgacaggcacggcgacccgggcacctgctgccgcgaccctgatcctccgccgtcgtcgggcaggttgcggtccccgcggttccctgcatctgttaagagggcgggggccgggaaggcggtgtccggggacggtgcccttgtcctcgaggcggccctggacgcgtcgcctgcggggcctgaaccgaggacccccccgtaccccgctttgtcctggtgaggcgctcctgccgcgcagcggcatcttcctctgttaacgtcgagaatgagtagtgaaggctgctcattatcctgcattgtaaagtaaaacttgtctttttagaagcgtctcctgaaaattggcggcatctcctgtgcttctctgggttgtattgggaaactgggccagtcgctgtcaggactgtcctgttggtaggtctcgagggaaagaaatgggagagaaacctaccccgtaaatattttcctactcaggtgcttttcgaactacctattaggtgattgcctttttttttttttttttttttcctgaacaaattacaaactgcaggtagaaatcaggaaggcctctttaggctcctcgccaggcagcctttttagcatttttctgaatgtgaacaggtccaaacgacagagcagaatttgaaaagaaaggttttgctgcagattcataacgaggactttatttttcattggtattttaatggtaatggtatagataatgttttatttttaatgttttttttaaatggctactaaaattttattttgaaatcacagctgtttcagtcacccccaagaaacctttaaaacgtcttattagaaacatttttatatataggaacatttagccatttatacacatttctaagatagaaatcacaatgaagatacgttgtttcactattccaagttccaagtaaccatggtgaatatggtttccccattccaaagtaaccattgtgaatgtggttatacacctccagtctttttctatgcagagcttaatacatgagtttttctaaagtgaattctcttttgtaattatctttttctgactagatagcatgccattatatggctgtaatattataatttatttcattggtcattctcagttcttccctattaaaatacattgttgggtaaatgtaaaagtccgcatatctatgattactttcgaatctcactctgtcgcccaggctggagtgtagtggcacgatctcggctcactgcaacctccacctcctgggttcaagtgattcccctgtgtcagcctcccgagtagctgggactacaggcgtgtgccaccatgcccggctaatttttgtatttttagtagagatggggtttcaccatatttgccaggctggtctcaaactcctgacctccagcgatctgcccgcctcggcctcgcaaagtgctgggattacaggtgtgagccaccgcgcccggcccatgattactttctaagtgtacattcctagatgatagaattattgggcagagtatgcacataagggtttttttttttttaatgtgaattgctcaatttccctctggaaaggatatgctgttttgcactcctaccaacaatctgtgagtgtccttacccacaccctagcaaaaggcaacatttaaagggatttctataggaatacttttgagccatacttgtactctccaaccaagtacaactacccctctctagtcatcacttttttcttatttgttgatagcacttcagcttctcatcttttattgcgcctttactattttggtaaagaaagcttatctaggtgaggcaaaggcgtaatagaacaaacaagagatttaaagttaggatccccttgaaatccagtatatctgattatcttgaacaagtcacttaacttttctaaggtactctttcctcagtgatgcattaggggttataatacagttgccttacttttcttaaggattattttaagggtcagttgaaacattcctggaaagtacatttagacacactttgtcttgttggtatttttaaaaattatttttaatttattatttttaaatagagacagtgtctcactatgttgcctaggctggtctcaaactcctgagctcgagcgattctcctgccttggccttgcagagtgctgggattacaggcatgagccactgcacccggccatgttattaccatctggtagaaatttcagagcacagaagcatcttgaggttcatacattttttaaagtaaataccttaattcaaaaggctattctctgataccctttgcccccatactctttcactccttaaatttgaagttttttgttttactgctgccctagattatattcaaaactttatcagggttatgaaattagatgagtgaatgaatctgcacaaatgttgaaggagcaaatatttaaaagcgctatataaagctgagagggacaggtataacgctaaataaaacacagtacttgggctcatgataaataaaacatggtctttgtgccctgcaggctacattatccaggcagcagagggcataaaagctgctttccaaatttagatggcaattttagatgtgatgcctgatgtcaactattctcaagtatgctaaagcccatttctgctgaccaagcacctgagcaagtgaccgggcagcctccacagggtcatctttctgaaggtaggattcgcaaggtgaggaagtggtagtattatagtccaagaacctgggttctactttcaggtcctccacctggggcagatcagttaacttttttgaggctggctttattcactcgtgaaatggaggtaattccagcttcctcatgtgtttgttgtgaggattacatgacatagtacgtgtacaacacttagtaaaccaggaactgctggtactcatgtcatcattcttacccactagagacttagcttccccaggaaagaaagtaggtctcttctaactttgtattcctaccccctagcagtcactggtacttagtcacgtttgataaattaacatagaatgtgaccactgataaaaattcagtttgggggtgccgggcacagtggctcacgcctgtaatcccagcacctttgggaagccaaggtggacggatcacctgaggtcaggagtttgagaccagcctggccaacatggtgaaaccccatctctgctaaaaatacaaaaagtagccaggcgtggtggtgcatgcctgtaatcccagctactcaggaggttgaggcatgagaattgcttgaacccaggaggcggaggttgcagtgagtcgagatcgtgccactgcactccagcctgggtgacagagcaagactctgtctctaaataaataagtaaattcagtttggacccaatgaagaaggtaatatgtaaatatggaatattgggaaaaattgaccacgttatttggagttaatgttaaggaatcaaaggaaacattgaactaattggacttagactcatctctgctatgactatcattctactttagatccctaatgccaggcaaatagtccctccttattaagaataaatgtactggctgggcatggtggttcatacttataatcccagcactttgggaggctgaactgggaggatcacctgaggccaggagtttgagactagccctggcaacatagcaagaccctgtctctacaaaaaaatttttaaaaaattagctgggtatggggtatgcacctatagtcccagctactcaaagactgaagcgagaggatcacttgagcccaggagttcaaggctgcagtgagctatgattgcactaccgcacaccagcctgggtgagggggtgagacctggactcaaggggaaaaaaaaaaaaaaaaaaaccgactcaaagtttctcccattttaaaacctcattcctagacctctaatcaccctgctgctacctagtttctctcttcccccttcccttcccagctggactgtttgaaagagttgttaataattggctttctcagtttagccctcaagacatgtgttrwsrgykkwcawwmctwktatkkgawctskctgcagtatttaccatgaaagtctccctttttgaaacatccttcttttgtttctgagattctgtgttccaaggttcccttcttcctcctgggaagttccttctcagtttctcctttgctggttcatcttcctccaaaggttgattttccgcaggcttgatccttcttgtgccgtactgtctctggaacaatttatagagcattgaggctatctcgtgtttgaagatttggtagaattccctgtgaaaccatctgggcctgacgctttttcatgtgatatttcctttataactttctcaatttttttttctgtggaaattggtctatttaagctttctaacatctaatggagaaaattttagtgatctgtacttttctagcaaattatccctttcatctggcgttttcaaatttatgttggtagaggtctacaaagacatcctttattatttttaaattttttcatcttatttcctccatgccctcattttgtgtttctgtgcttttccccttttttccttaagttatctaatggtttatctattttattttgttatttttttcaaaaactacagaattttgaattattaatwagatctgtttttctgtttttttcccctcattcttttcttctactttcatctttttttgcttgttttttcctgagacaaggtctctctctgtcactcagactgacgtgcagtcagtggcattgtctcagctcactgtagcctctacctcctgggctcaagcaatcctcccacctcagcctcccaagtagctgggactacagtggtgcaccaccacacttggctaattttgataatttttttttgtagagacaaggtctgcccatgttgcccgggctggccttgaactcttggcctcaaataatcctcctgcctcagtctcccaaagtgccggaattacaggcatgagccactgtgcctggcctactttaatctttattatttacttccatgtcctttcttttgg

SEQ ID NO: 5 - DNA, 1793 bp, Homo sapiens
gcttcccggccgggtgagcggctcggggggaggcggggcgaggaggcctgggcccgcagggagggcgtgtccgccacccaggaggggcggttctcggggtggcggatggggcagcggggaggatgcggcggtccgactcgggttctgttgggacagctcctctgcgccccgtaggtccctcggtcagcgccttcccttgggcgcggcgacccggcgcgggtttccaggggacagctgcccggcgcccctgaggtccctcgacaggcacggcgacccgggcacctgctgccgcgaccctgatcctccgccgtcgtcgggcaggttgcggtccccgcggttccctgcatctgttaagagggcgggggccgggaaggcggtgtccggggacggtgcccttgtcctcgaggcggccctggacgcgtcgcctgcggggcctgaaccgaggacccccccgtaccccgctttgtcctggtgaggcgctcctgccgcgcagcggcatcttcctctgttaacgtcgagaatgagtagtgaagactgctcattatcctgcattgtaaagtaaaacttgtctttttagaagcgtctcctgaaaattggcgcgtctcctgtgcttctctgggttgtattgggaaactgggccagtcgctgtcaggactgtcctgttggtaggtctcgagggaaagaaatgggagagaaacctaccccgtaaatattttcctactcaggtgcttttcgaactacctattaggtgattgccttttttttttttttcttttttttcctggaacaaattacaaactgcaggtagaaatcaggaaggcctctttaggctcctcgccaggcagcctttttagcatttttctgaatgtgaacaggtccaaacgacagagcagaatttgaaaagaaaggttttgctgcagattcataacgaggactttatttttcattggtattttaatggtaatggtatagcaatgttttatttttaatgttttttttaaatggctactaaaattttattttgaaatcacagctgtttcagtcacccccaagaaacctttaaaacgtcttattagaaacatttttatatataggaacatttagccatttatacacatttctaagatagaaatcacaatgaagatacgttgtttcactattccaagttccaagtaaccatggtgaatatggtttccccattccaagtaaccattgtgaatgtggttatacacctccagcctgggtgacagagcaagactctgtctctaaataaataagtaaattcagtttggaccaatgaagaaggtaatatgtaaatatggaatattgggaaaaattgaccacgttatttggagttaatgttaaggaatcaaaggaaacattgaactaatcggacttagactcatctctgctatgactatcattctactttagatccctaatgccaggcaaatagtccctccttattaagaataaatgtactggctgggcatggtggttcatacttataatcccagcactttgggaggctgaactgggaggatcacctgaggccaggagtttgagactagccctggcaacatagcaagaccctgtctctacaaaaaaatttttaaaaaattagctgggtatggggtatgcacctatagtcccaggctactcaaagactgaagcgagaggatcacttgagcccaggagttcaaggctgcagtgagctatgattgcactaccgcacaccagcctgggtgagggggtgagacctggactcaaggggaaaaaaaaaaaaaaaaaaa

SEQ ID NO: 6 - DNA, 19 bp, Artificial Sequence
  Description of Artificial Sequence Primer
ggattacaggyrtgagcca

SEQ ID NO: 7 - DNA, 19 bp, Artificial Sequence
  Description of Artificial Sequence Primer
rccaytgcactccagcctg

SEQ ID NO: 8 - DNA, 21 bp, Artificial Sequence
  Description of Artificial Sequence Primer
ggttctcggggtgggggatgg

SEQ ID NO: 9 - DNA, 21 bp, Artificial Sequence
  Description of Artificial Sequence Primer
gacgttaacagaggaagatgc

SEQ ID NO: 10 - DNA, 24 bp, Artificial Sequence
  Description of Artificial Sequence Primer
gaggcggcggcagagtgaggggcg

SEQ ID NO: 11 - DNA, 24 bp, Artificial Sequence
  Description of Artificial Sequence Primer
gccccatccgccaccccgagaacc

SEQ ID NO: 12 - DNA, 21 bp, Artificial Sequence
  Description of Artificial Sequence Probe
cggcggcggcggcggcggcgg

SEQ ID NO: 13 - DNA, 51 bp, Artificial Sequence
  Description of Artificial Sequence Synthetic oligonucleotide
ccgccgccgccgccgccgccgccgccgccgccgccgccgccgccgccgccg

SEQ ID NO: 14 - DNA, 45 bp, Artificial Sequence
  Description of Artificial Sequence Synthetic oligonucleotide
cggcggcggcggcggcggcggcggcggcggcggcggcggcggcgg

(19) United States
(12) Patent Application Publication
(10) Pub. No.: US 2005/0112613 A1
(43) Pub. Date: May 26, 2005

(54) Methods and reagents for predicting the likelihood of developing short stature caused by FRAXG

(75) Inventors: Ralf Krahe, Bellaire, TX (US); Shanxiang Zhang, Binhai County (CN); Albert de la Chapelle, Delaware, OH (US)

Correspondence Address: CALFEE HALTER & GRISWOLD, LLP, 800 SUPERIOR AVENUE, SUITE 1400, CLEVELAND, OH, 44114, US

(73) Assignee: The Ohio State University Research Foundation, Columbus, OH (US)

(21) Appl. No.: 10/831,819

(22) Filed: Apr. 26, 2004

Related U.S. Application Data
(60) Provisional application No. 60/320,146, filed on Apr. 25, 2003.

(51) Int. Cl. C12Q 1/68; C07H 21/04

(52) U.S. Cl. 435/6; 536/24.3; 435/325

(57) ABSTRACT

The invention provides methods for identifying an infant or child predisposed to develop symptoms of short stature, or an adult capable of genetically transmitting a predisposition to develop short stature to an offspring. The methods comprise analysis of a region of DNA in the genome of a subject located at or near a site called FRAXG on Xp22.1. In one embodiment, the analysis comprises determining the number of (CGG)ₙ/(CCG)ₙ nucleotide triplets within FRAXG. In another embodiment, the analysis comprises determining whether there is hypermethylation within the CpG island encompassing FRAXG. The invention also comprises probes and primers for use in the above analyses, kits containing the probes and/or primers for performing the analyses, and cell lines containing high numbers of (CGG)ₙ/(CCG)ₙ nucleotide triplets within FRAXG.

### CROSS REFERENCE TO RELATED APPLICATIONS

This application claims priority to U.S. Provisional Application No. 60/320,146, filed Apr. 25, 2003, which is incorporated herein by reference in its entirety.

### GOVERNMENT RIGHTS

This invention was supported, at least in part, by grant P30 CA16058 from the National Cancer Institute. The Federal Government may have certain rights in this invention.

### FIELD OF THE INVENTION

The invention relates to methods, reagents and kits for determining whether an individual has a predisposition to develop short stature or is capable of genetically transmitting such predisposition to an offspring.

### BACKGROUND

Short stature is defined as a condition in which the height of an individual is at least two standard deviations below the corresponding mean height for a given age, sex and population group. It affects about 3% of the population and symptoms are not usually apparent at birth, but at sometime thereafter. While environmental, physiological and genetic factors may contribute to some instances of the condition, the majority of cases of the condition do not have any known etiology. Such occurrences of the condition are called “idiopathic short stature.”

Some cases of short stature in females are associated with Turner syndrome, a syndrome in which females are missing an X chromosome (XO females). While the majority of fetuses with a single X chromosome do not survive to term, some do survive. In these survivors, it has been found that some portions of the X chromosome remain (i.e., there is not a complete loss of one X chromosome).

Based on the absence of at least some parts of the X chromosome in Turner syndrome, some researchers have hypothesized that short stature is due to the loss of gene function, specifically on the X chromosome. However, due to the large size of deletions (i.e, many genes deleted) in Turner syndrome patients, it has not been possible to identify candidate genes or genetic regions that are generally responsible for short stature.

There is a need to identify the genetic regions, and alterations therein, involved in short stature in individuals of both genders, and to develop reagents and assays to perform identification assays. Such identification assays would be useful in diagnosis of short stature in individuals who present with symptoms. Such assays and reagents would also be useful in identifying fetuses, infants and children with no symptoms, but who are genetically predisposed to develop symptoms of the condition in the future. Such predisposed fetuses, infants and children, and their parents, may want to know that they are so predisposed in order to plan to begin therapeutic treatment for the condition. It would also be useful to identify adults who may genetically pass to their offspring a predisposition to develop the condition. Such adults may want to know that they could transmit the trait before deciding to have a child.

### SUMMARY OF THE INVENTION

We have discovered a chromosomal region in Xp22.1 containing a new rare heritable, folate-sensitive fragile site (RHFFS). The new RHFFS is part of a CpG island and is part of or near a transcriptional promoter/regulatory region. There is a transcribed region located immediately centromeric (i.e. toward the chromosome centromere) of the new RHFFS. We have named the new RHFFS, FRAXG, and have discovered that it is polymorphic in that the number of (CGG)ₙ/(CCG)ₙ nucleotide triplets within FRAXG is highly variable between individuals.

We have found that some individuals with numbers of (CGG)ₙ/(CCG)ₙ nucleotide triplets in FRAXG that are very much higher than the average number of (CGG)ₙ/(CCG)ₙ nucleotide triplets in members of the population at large are more likely to develop symptoms of short stature than individuals with numbers of (CGG)ₙ/(CCG)ₙ nucleotide triplets near the population average (i.e., a population of “normal” individuals, not having or predisposed to having short stature). We have also found that the FRAXG-containing CpG island is hypermethylated in individuals with the higher than average number of (CGG)ₙ/(CCG)ₙ nucleotide triplets within FRAXG.

The invention provides for methods for diagnosing short stature in individuals who present with symptoms. The invention also provides methods for identifying a fetus, infant or child with no symptoms who is predisposed to develop symptoms in the future, and for identifying adults who may genetically pass to their offspring a predisposition to develop the condition. The methods are based on analysis of the polynucleotide sequence in the FRAXG region and surrounding chromosomal regions. One method comprises determining the approximate number of (CGG)ₙ/(CCG)ₙ nucleotide triplets within one or more alleles of FRAXG of a subject and comparing the number of triplets in said one or more alleles with the number of triplets found in the general population, and more particularly, the population of which the individual is a member. Another method comprises determining the presence and extent of methylation or hypermethylation of cytosine nucleotides that are part of CpG dinucleotides within the CpG island that encompasses FRAXG.

The invention also provides reagents, including specifically nucleotide probes and primers, for use in the above described assays. The invention also provides kits for performing the above described assays. The invention also provides cell lines from individuals with increased numbers of (CGG)ₙ/(CCG)ₙ nucleotide triplets within FRAXG. Such cell lines are useful for providing FRAXG chromosomal regions of known sizes for use as standards when assaying DNA from individuals for amplification of FRAXG.

### BRIEF DESCRIPTION OF THE DRAWINGS

The present invention may be more readily understood by reference to the following drawings wherein:

FIG. 1. A shows the pedigree of the Finnish family. The proband (i.e., the initial individual studied) is indicated by the arrow. The filled circles indicate females in the pedigree that have the FRAXG site. The stated percentages indicate the expression frequencies of FRAXG under the folate-sensitive fragile site culture conditions. No one else in the pedigree besides the proband displayed the short stature phenotype. B shows the growth curve of the proband over approximately the first 15 years of life (heavy line). Arrows indicate the period that the child received growth hormone treatment. A mean growth curve as well as growth curves showing two standard deviations above the mean and two standard deviations below the mean are shown. C shows Giemsa staining of the proband's partial metaphase spreads showing FRAXG as a nonstaining gap (arrow). D shows Trypsin-Giemsa staining that locates FRAXG to Xp22.1 (arrow).

FIG. 2. Giemsa staining of metaphase spreads from the proband's lymphoblastoid cell line, cultured under folate-sensitive fragile site inducing conditions, showing FRAXG as a chromatid break (A) and a nonstaining gap (B) (indicated by arrows).

FIG. 3. Fluorescence in situ hybridization (FISH) mapping of FRAXG with YAC y827E10. The FRAXG was shown as a chromatid break indicated by the arrow (1). YAC y827E10 was located on the gap (arrow, 2). CEP-X, a marker for X-chromosome centromere, and BAC b733018, located on Xp22.31, were included as the control for the X-chromosome centromere and telomere, respectively.

FIG. 4. FISH mapping of FRAXG with YACs y911G5 and y946F5. y911G5 was located telomeric to FRAXG (A), and y946F5 centromeric to FRAXG (B) (indicated by arrows). CEP-X, a marker for the X-chromosome centromere, and b733018, located on Xp22.31, were included as the controls for the X-chromosome centromere and telomere, respectively.

FIG. 5. Mapping of FRAXG to a critical region of about 1 Mb in Xp22.1 by FISH with a contig of six YAC clones. The numbers in brackets are the estimated sizes in kb for the YACs. Cen: centromeric to FRAXG; Tel: telomeric to FRAXG; N.D.: not done.

FIG. 6. Mapping of FRAXG to a critical region of less than 200 kb in Xp22.1 by FISH with a contig of 23 BAC clones. These clones represent the minimal tiling path of this region. The critical region of FRAXG is indicated by the solid bar. All BACs right to b228D12 (including 228D12) are located centromeric to FRAXG, and those left to b692N21 (including 692N21) are located telomeric to FRAXG. BAC b393H10 is located right on the gap.

FIG. 7. FISH mapping of FRAXG with BAC b393H10. The FRAXG was shown as a non-staining gap indicated by the arrow. BAC b393H10 was located right on the gap (indicated by arrow). CEP-X, a marker for X-chromosome centromere, and BAC b733018, located on Xp22.31 were included as the control for the X-chromosome centromere and telomere respectively.

FIG. 8. Detection of (CGG)ₙ/(CCG)ₙ-positive fragments in b1139J14, b1037J10, and b393H10 by Southern blot. The probe was [γ-³²P] ATP-labeled (CCG)₇.

FIG. 9. A shows a Southern blot analysis of b393H10 with [γ-³²P] ATP-labeled (CCG)₇ as the probe. B shows a restriction map of the region around the (CCG)₁₇ repeat. H, HindIII; N, NotI; RI, EcoRI; RV, EcoRV. Also shown is the 770 bp HpaI-EcoRI fragment (HpRI), which does not contain the (CCG)₁₇. This fragment was used as a probe in some Southern blotting studies described in this application.

FIG. 10. Shows a sequence that is SEQ ID NO. 1. The (CGG)ₙ/(CCG)ₙ repeat (bold type) and part of its flanking sequence from b393H10 is shown. The underlined sequences are locations for PCR primers, forward primer 393H10_F (SEQ ID NO. 2) and reverse primer 393H10_R (SEQ ID NO. 3), used in the (CGG)ₙ/(CCG)ₙ repeat copy number analysis.

FIG. 11. A distribution analysis of polymorphic FRAXG (CGG)ₙ/(CCG)ₙ triplet numbers in normal Finnish population by PCR across the repeat. A total of 286 randomly selected normal Finnish males were analyzed.

FIG. 12. A genomic DNA Southern blot showing expansion in FRAXG-expressing individuals is shown. Genomic DNA was digested by EcoRI, and the samples were hybridized to the 0.77 kb HpaI-EcoRI fragment (HpRI).

FIG. 13. Expansions in FRAXG-positive individuals by Southern analysis and methylation analysis of FRAXG CpG island with probe HpRI is shown. Genomic DNA from Epstein-Barr virus-transformed cell lines established from the Finnish FRAXG family and CEPH family GM10859 (lane 1) and GM17057 (lane 7) was subject to either HindIII single digestion or HindIII plus NotI double digestion. A control probe from 11q22 was used as the digestion and load control.

FIG. 14. Shown is the sequence of the 6,882 base pair genomic sequence (SEQ ID NO. 4). The sequence shown in FIG. 10 is within the 6,882 base pair sequence and is shaded. The (CGG)ₙ/(CCG)ₙ repeat region is shown within the shaded region in bold type. The (CGG)ₙ/(CCG)ₙ region shown here has 15 repeats, rather than the 17 repeats in FIG. 10.

FIG. 15. A shows the predicted promoter and CpG island as well as the (CGG)ₙ/(CCG)ₙ repeat along the length of the genomic DNA. The two solid bars below the line indicating the genome, are the two exons transcribed from the sequences. B is a similar drawing showing the genomic DNA. The dark barred areas on the genomic DNA show the exonic regions. The RNA derived from the two exons (indicated as FXGC) is shown as a bar below the genome region.

FIG. 16. The figure shows the sequence of the 1,793 base pair transcript from the FRAXG region (FXGC) (SEQ ID NO. 5).

FIG. 17. The figure shows a multiple tissue Northern blot probed by G1Ex1, indicating FXGC expression in different tissues. Tissues: 1, brain; 2, heart; 3, skeletal muscle; 4, colon; 5, thymus; 6, spleen; 7, kidney; 8, liver; 9, small intestine; 10, placenta; 11, lung; 12, peripheral blood lymphocytes; 13, stomach; 14, thyroid; 15, lymph node; 16, trachea; 17, adrenal gland; 18, bone marrow. β-actin was used as an internal control for the comparison.

### DETAILED DESCRIPTION OF THE INVENTION

Definitions

Herein, “predisposition to develop short stature,” is used to refer to infants or children who have a significant likelihood of developing symptoms of short stature condition at some time in the future. Such significant likelihood of developing the symptoms encompasses a range of probabilities that the individual is likely to develop such symptoms. At the low end, the probability of developing the symptoms is any probability that is higher than the average probability of a population of individuals not having or not being predisposed to develop symptoms of short stature (see “normal individuals” below). At the high end, the probability of developing the symptoms is 1.0 or 100%.

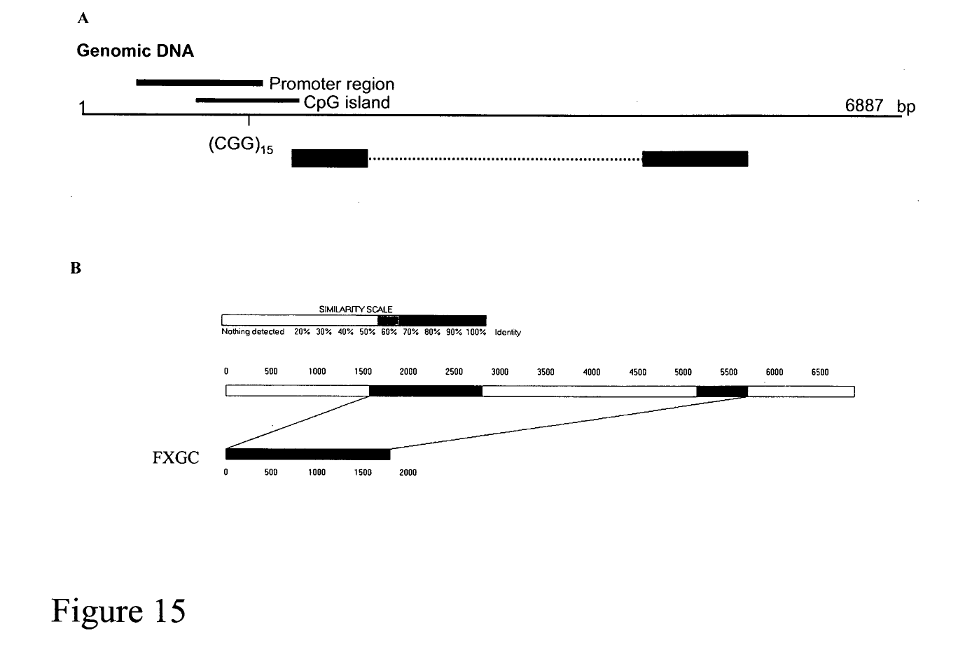

Herein, “CpG island” means an area of a genome that is greater than approximately 60% in G+C content. The specific CpG island referred to in this application contains or encompasses the FRAXG site, meaning that the FRAXG site is bounded on either side by regions of genome sequence that, together with FRAXG, comprise sequences greater than approximately 60% in G+C content (see FIG. 15A).

Herein, the “the FRAXG CpG Island” refers to the CpG island which comprises FRAXG, and which is bounded on either side by regions of genome sequence that, together with FRAXG, comprise sequences greater than approximately 60% in G+C content.

Herein, “(CGG)ₙ/(CCG)ₙ” refers to the nucleotide triplet within the chromosomal region Xp22.1 that is present in various numbers in different individuals and which identifies FRAXG. The designation indicates that on one strand of the genomic DNA, the sequence is 5′-CCG-3′ while the complementary strand of the DNA is 5′-CGG-3′. In some individuals, the triplet repeats are perfect repeats in that no sequences other than repeating sequences of CCG are present (i.e., contiguous CCG repeats). In other cases, the tandem CCG repeats may be interrupted by one or more sequences that are not CCG (i.e., noncontiguous CCG repeats). In other words, the sequence of FRAXG may not be a perfect tandem repeat of CCG in all individuals.

Herein, “normal individuals” or “normal population of individuals” refers to adult individuals or a group of adult individuals that do not have symptoms of short stature and do not have family members that have symptoms of short stature. Such individuals do not display elevated numbers of (CGG)ₙ/(CCG)ₙ nucleotide triplets in the FRAXG CpG Island.

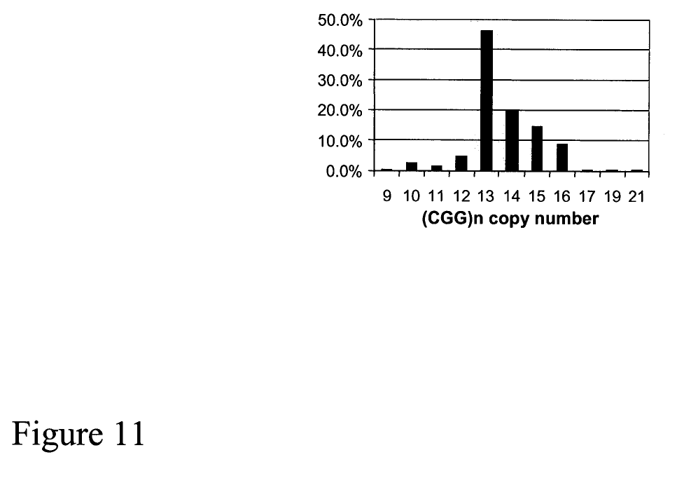

Herein, an “unelevated number” of (CGG)ₙ/(CCG)ₙ triplets is a number of triplets found in a normal population of individuals. This number will vary depending on the human population from which individuals are chosen. Determination of whether a number of triplets in an individual is unelevated is made based on a distribution of numbers of (CGG)ₙ/(CCG)ₙ triplets in multiple, normal individuals of the population. For example, the data in FIG. 11 show that normal individuals from a particular Finnish population have from between 9 to 21 triplets in their FRAXG CpG Island.

Herein, an “elevated number” of (CGG)ₙ/(CCG)ₙ triplets is a number that is more than the number found in normal individuals. Such a number of triplets can be said to be “significantly greater” than the number found in normal individuals. Such an elevated number of repeats refers to a number of repeats that is higher, based on statistical significance, than the average number from a normal population, using standard statistical methods. For example, a proband from the Finnish population had at least 500 (CGG)ₙ/(CCG)ₙ nucleotide triplets.

Herein, “methylation” or “methylated” refers to 5-methylcytosine in the genome of a subject, as compared to cytosine, which is not methylated. Cytosines that are methylated are part of 5′-CpG-3′ dinucleotides within a genome.

Herein, “hypermethylated” refers to a condition where a cytosine within a CpG dinucleotide within a genome of a first individual is methylated to 5-methylcytosine and where the corresponding cytosine in the genome of a second individual is not methylated. At the particular region of the genome where the specific CpG dinucleotide is present, the genome of the first individual is said to be hypermethylated as compared to the second individual. Herein, the region of the genome in which detection of 5-methylcytosines is relevant is the region comprising the FRAXG CpG Island.

Herein, “proband” refers to an affected person with a genetic disorder ascertained independently of his or her relatives in a genetic study.

The invention relates to methods for diagnosing an individual as having short stature. The invention also relates to methods for identifying individuals, particularly fetuses, infants and children that are predisposed to developing symptoms of short stature in the future based on FRAXG. The invention also relates to methods for identifying individuals that are capable of genetically transmitting predisposition to develop short stature to their offspring. In one embodiment, the method is directed toward assaying a sample of DNA from an individual for the number of (CGG)ₙ/(CCG)ₙ nucleotide repeats within FRAXG, a newly discovered RHFFS within chromosomal region Xp22.1. The presence of a number of (CGG)ₙ/(CCG)ₙ nucleotide triplet repeats in one or both alleles of the individual that is significantly greater than the average number of repeats in a population of normal individuals indicates the individual either has short stature, is predisposed to developing symptoms of short stature in the future, or is capable of genetically transmitting the predisposition to offspring. In another embodiment, the method is directed toward assaying a sample of DNA from an individual for the presence of 5-methylcytosines within the FRAXG CpG Island. The presence of hypermethylated regions indicates the individual either has short stature, is predisposed to develop symptoms of short stature in the future, or is capable of transmitting the predisposition to offspring.

The invention also relates to reagents (e.g., probes and primers) for use in practicing the invention. The invention also relates to kits containing the reagents for use in the inventive methods. The invention also relates to cell lines from individuals with increased numbers of (CGG)ₙ/(CCG)ₙ nucleotide triplets within FRAXG, which cell lines are useful for providing controls in determining (CGG)ₙ/(CCG)ₙ nucleotide triplet copy number and methylation state.

Human Chromosomal Fragile Sites

Chromosomal fragile sites are regions of chromosomes that show an increased frequency of gaps and breaks when cells from which the chromosomes are prepared are exposed to specific conditions of tissue culture or chemical agents. Although initially observed in cells grown in culture, it is believed that at least some of the fragile sites detected in cultured cells are indicative of regions of chromosomes that are unstable and that this instability may be mechanistically involved in human mutations.

Based on their frequency in cultured cells, fragile sites are classified as common or rare. Common fragile sites are present probably on all chromosomes, which is part of normal chromosome structure. Rare fragile sites vary in frequency from only a handful of reports to 1 in 40 chromosomes.

There are more than 80 common fragile sites reported to date. Based on the conditions of tissue culture required to induce their cytogenetic expression, common fragile sites are further divided as aphidicolin inducible, 5-azacytidine inducible, and bromodeoxyuridine inducible. The molecular basis for these sites is not yet understood. Common fragile sites have been proposed to be involved in chromosomal deletions, rearrangements, and to be the preferential site of viral integration. Some common fragile sites have been observed in solid tumors including breast, lung, head and neck, and cervical cancers.

Generally, common fragile sites seem to be large fragile regions, spanning from ˜150 to over 1000 kb in size. Sequence analyses and comparisons of the regions for these four cloned common fragile sites have indicated that those regions tend to be AT-rich in sequence and show high-flexibility, low-stability, and may form unusual DNA structures. However, no special sequences, such as expanded microsatellite repeats identified in the rare fragile sites, have been identified.

Rare fragile sites are of various types. They are divided into folate sensitive, distamycin A inducible, and bromodeoxyuridine requiring fragile sites. There are more than 25 reported to date. Based on molecular characterization, five of them are heritable folate-sensitive fragile sites. These are all caused by expansion of a normally polymorphic (CGG)ₙ/(CCG)ₙ trinucleotide repeat. Two other of these sites are distamycin A inducible and bromodeoxyuridine requiring fragile sites, both caused by expansion of AT-rich minisatellite repeats.

Certain folate-sensitive fragile sites have been linked to clinical phenotypes. FRAXA is linked to fragile X syndrome; the most common inherited mental retardation in children. Fragile X syndrome is caused by a functional deficiency of the FMR1 gene. More than 95% of this deficiency is caused by an expansion of an unstable (CGG)ₙ/(CCG)ₙ trinucleotide repeat in the 5′ UTR region of FMR1 gene. The expansion of the (CGG)ₙ/(CCG)ₙ repeat induces the hypermethylation of itself and an adjacent CpG island, which results in downregulation of transcription of FMR1. The presence of expanded (CGG)ₙ/(CCG)ₙ in the mutant FMR1 transcripts can also interfere with the translation of FMR1. Similarly, FRAXE is linked to a nonspecific mild mental retardation due to transcriptional downregulation of the FMR2 gene, and FRA11B is caused by an expansion of a (CGG)ₙ/(CCG)ₙ repeat in the 5′UTR of proto-oncogene CBL2, which may be involved in Jacobsen syndrome.

Methods for Determining the Number of (CGG)ₙ/(CCG)ₙ Repeats within FRAXG

Determining Whether one or More FRAXG Alleles Have Normal, Un-Elevated Numbers of (CGG)ₙ/(CCG)ₙ Repeats

In normal individuals, both FRAXG alleles have unelevated numbers of (CGG)ₙ/(CCG)ₙ repeats. Because of the polymorphic nature of FRAXG, however, the two alleles are unlikely to have the same number of (CGG)ₙ/(CCG)ₙ repeats. Therefore, the two FRAXG alleles, even in normal individuals, are likely to be different in size.

The preferred method for detecting elevated numbers of (CGG)ₙ/(CCG)ₙ nucleotide triplets within FRAXG of a subject is a two-step method that takes advantage of the fact that the FRAXG alleles in normal individuals are likely to be of different sizes. In the first step, a determination is made of the number of FRAXG alleles present that contain unelevated numbers of (CGG)ₙ/(CCG)ₙ repeats. One such method uses amplification of a sample of DNA from the subject using the polymerase chain reaction (PCR) and nucleotide primers that direct amplification of FRAXG alleles. Such primers are described below, but generally hybridize to a genomic region on either side of FRAXG and direct PCR amplification across the FRAXG region. Such primers are able to amplify the FRAXG region if FRAXG contains an unelevated number of (CGG)ₙ/(CCG)ₙ triplets. The size of the amplified fragment is indicative of the number of (CGG)ₙ/(CCG)ₙ repeats within FRAXG. As the number of (CGG)ₙ/(CCG)ₙ triplets within a FRAXG allele increases, however, the ability of the primers to direct amplification across the FRAXG region decreases. The finding is that when FRAXG approaches a size such that an individual having that allele in their genome is predisposed to develop symptoms of short stature, the PCR is not able to amplify across the FRAXG region.

The results of the PCR step, therefore, indicate whether the DNA from the individual has one or two FRAXG alleles containing an unelevated number of (CGG)ₙ/(CCG)ₙ triplets. If two amplified products result from the PCR step (each representing amplification of a different-sized FRAXG allele), the conclusion generally is that DNA from the individual has two FRAXG alleles, both containing an unelevated number of (CGG)ₙ/(CCG)ₙ triplets. If one amplified product results from the PCR step, the conclusion generally is that DNA from the individual has one FRAXG allele containing an unelevated number of (CGG)ₙ/(CCG)ₙ triplets (the unelevated allele is the template for the PCR product) and one FRAXG allele containing an elevated number of triplets. If no amplified product results from the PCR step, the conclusion generally is that DNA from the individual has no FRAXG alleles containing an unelevated number of (CGG)ₙ/(CCG)ₙ triplets and that both alleles contain an elevated number of triplets.

Determining the Number of (CGG)ₙ/(CCG)ₙ Repeats for FRAXG Alleles

If the results from the first PCR step indicate that DNA from an individual has one or more FRAXG alleles with elevated numbers of (CGG)ₙ/(CCG)ₙ triplets, the second step of the method is preferably performed. In the second step, the DNA from the individual is analyzed using a method that detects the size of the FRAXG allele. One method of doing this is using Southern blotting to determine the approximate number of (CGG)ₙ/(CCG)ₙ nucleotide triplets within the FRAXG alleles, specifically within the one or more FRAXG alleles that containing elevated numbers of (CGG)ₙ/(CCG)ₙ triplets.

To begin the analysis and, specifically, to perform the first step which is PCR amplification of the FRAXG region, genomic DNA is isolated from cells from the subject. Any such cells that contain chromosomes can be used. In order to isolate the DNA, the cells are obtained or isolated. Commonly, DNA is obtained from cells from peripheral blood. Whole blood or a cellular fraction (e.g., leukocytes) can be used. For example, a cellular fraction can be prepared as a “buffy coat” (i.e., leukocyte-enriched blood portion) by centrifuging 5 ml of whole blood for 10 min at 800 times gravity at room temperature. Red blood cells sediment most rapidly and are present as the bottom-most fraction in the centrifuge tube. The buffy coat is present as a thin creamy white colored layer on top of the red blood cells. The plasma portion of the blood forms a layer above the buffy coat. Fractions from blood can also be isolated in a variety of other ways. One method is by taking a fraction or fractions from a gradient used in centrifugation to enrich for a specific size or density of cells. Another preferred cell type from which to obtain DNA is from a scraping of cheek cells from the individual.

Once the cells have been obtained or isolated, DNA is then isolated from the cells. Procedures for isolation of DNA from such cell samples are well known to those skilled in the art. Commonly, such DNA isolation procedures comprise lysis of cells present in the samples using detergents, for example. After cell lysis, proteins are commonly removed from the DNA using various proteases. RNA is removed using RNase. The DNA is then commonly extracted with phenol, precipitated in alcohol and dissolved in an aqueous solution.

FRAXG Primers

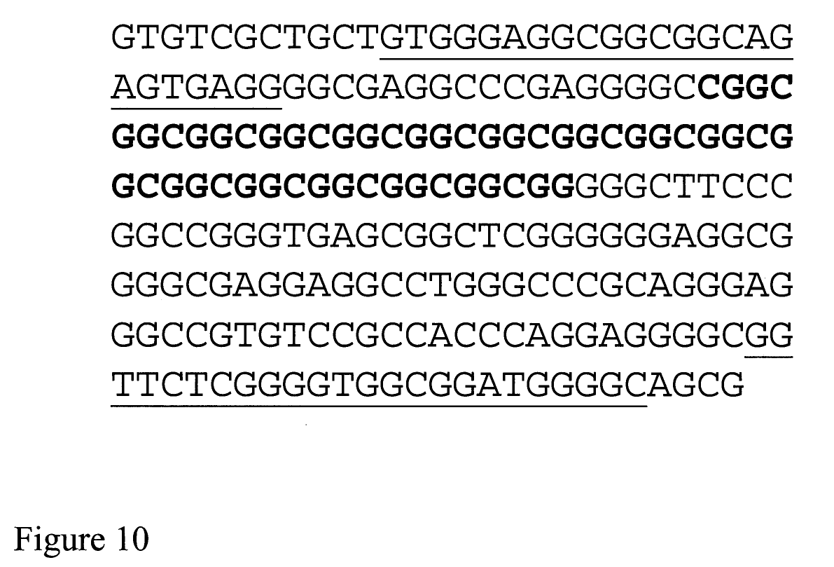

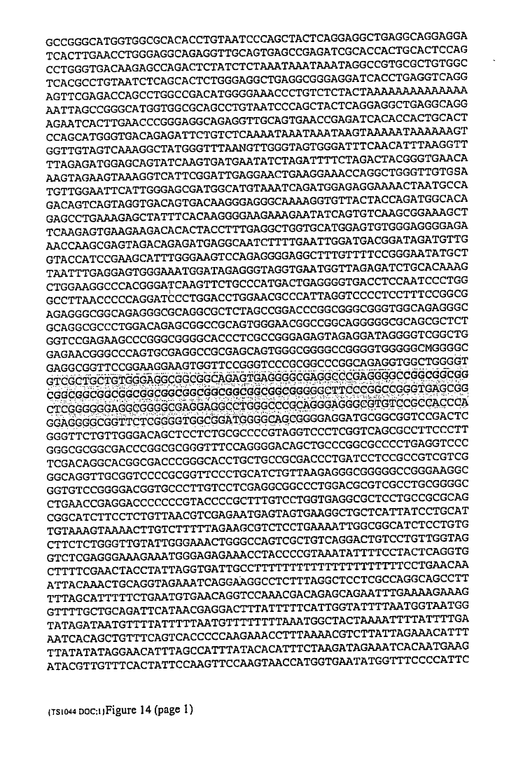

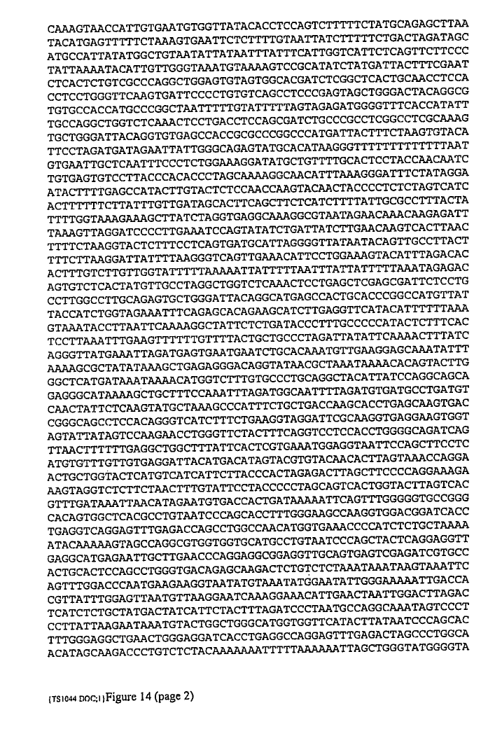

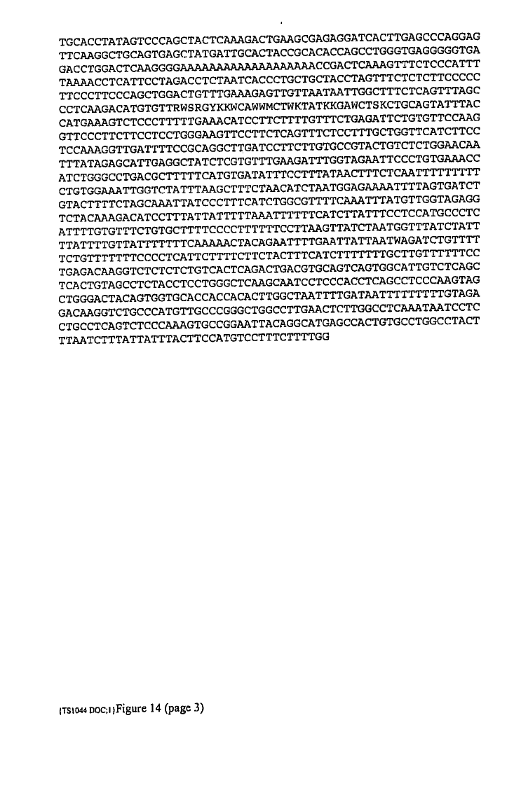

To use PCR to amplify the FRAXG region, the DNA isolated from the cells of the individual is amplified using two PCR primers that hybridize to regions that span, flank or are located on either side of FRAXG. The regions to which the two PCR primers should hybridize can be determined from examination of the nucleotide sequence flanking the FRAXG region containing the triplet repeats (see FIGS. 10 and 14).

Such primers will normally be between 10 to 30 nucleotides in length and have a preferred length from between 18 to 22 nucleotides. One primer is called the “forward primer” and is located at the left end of the FRAXG region. The forward primer is identical in sequence to a region in the top strand of the DNA (i.e., when a double-stranded DNA is pictured using the standard convention where the top strand is shown with polarity in the 5′ to 3′ direction). The sequence of the forward primer is such that it hybridizes to the strand of the DNA which is complementary to the top strand of DNA. The other primer is called the “reverse primer” and is located at the right end of the FRAXG region. The sequence of the reverse primer is such that it is complementary in sequence to a region in the top strand of the DNA. The reverse primer hybridizes to the top strand of the DNA PCR primers are also chosen subject to a number of other conditions. PCR primers should be long enough (preferably 10 to 30 nucleotides in length) to minimize hybridization to greater than one region in the template. Primers with long runs of a single base should be avoided, if possible. Primers should preferably have a percent G+C content of between 40 and 60%. If possible, the percent G+C content of the 3′ end of the primer should be higher than the percent G+C content of the 5′ end of the primer. Primers should not contain sequences that can hybridize to another sequence within the primer (i.e., palindromes). Two primers used in the same PCR reaction should not be able to hybridize to one another. Although PCR primers are preferably chosen subject to the recommendations above, it is not necessary that the primers conform to these conditions. Other primers may work, but have a lower chance of yielding good results.

PCR primers that can be used to amplify DNA within a given sequence are preferably chosen using one of a number of computer programs that are available. Such programs choose primers that are optimum for amplification of a given sequence (i.e., such programs choose primers subject to the conditions stated above, plus other conditions that may maximize the functionality of PCR primers). One computer program is the Genetics Computer Group (GCG recently became Accelrys) analysis package which has a routine for selection of PCR primers. There are also several web sites that can be used to select optimal PCR primers to amplify an input sequence. One such web site is http://alces.med.umn.edu/rawprimer.html. Another such web site is http://www-genome.wi.mit.edu/cgi-bin/primer/primer3_www.cgi.

One primer is located on either side of the FRAXG region. Good results for amplification of the FRAXG region have been obtained using a forward primer, (SEQ ID NO. 2), of sequence 5′-GTGGGAGGCGGCGGCAGAGTGAGG-3′ and a reverse primer, (SEQ ID NO. 3), of sequence 5′-GCCCCATCCGCCACCCCGAGAACC-3′. Another primer set giving good results is 5′-GAGGCGGCGGCAGAGTGAGGGGCG-3′ (SEQ ID NO. 10) and 5′-GCCCCATCCGCCACCCCGAGAACC-3′ (SEQ ID NO. 11). Many other primer pairs are possible as long as one primer is designed to hybridize to a nucleotide sequence to the left of FRAXG and the other primer is designed to hybridize to a nucleotide sequence to the right of FRAXG and the nucleotide distance between the two primers is such that amplification of a FRAXG allele containing an unelevated number of (CGG)ₙ/(CCG)ₙ repeats is not so great that amplification cannot occur. Preferably, the primers are also selected based on the other characteristics discussed above.

PCR Amplification

Once the forward and reverse PCR primers are determined, they are mixed with the genomic DNA and the PCR amplification reaction is performed. A standard PCR reaction contains a buffer containing 10 mM Tris-HCl (pH 8.3), 50 mM KCl, and 6.0 mM MgCl₂, 200 uM each of dATP, dCTP, dTTP and dGTP, two primers of concentration 0.5 uM each, 7.5 ng/ul concentration of template cDNA and 2.5 units of Taq DNA Polymerase enzyme. Variations of these conditions can be used and are well known to those skilled in the art.

The PCR reaction is preferably performed under high stringency conditions. Such conditions are equivalent to or comparable to denaturation for 1 minute at 95° C. in a solution comprising 10 mM Tris-HCl (pH 8.3), 50 mM KCl, and 6.0 mM MgCl₂, followed by annealing in the same solution at about 62° C. for 5 seconds.

The products of the PCR reaction can be detected in various ways. One way is by agarose gel electrophoresis which involves separating the DNA in the PCR reaction by size in electrophoresis. The agarose gel is then stained with dyes that bind to DNA and fluoresce when illuminated by light of various wavelengths. Preferably the dye used is ethidium bromide and the illumination uses an ultraviolet light.

Determining the Number of (CGG)ₙ/(CCG)ₙ Repeats

Since the PCR promoters are chosen to flank or span the region of FRAXG containing the triplet repeats, the size of the amplified DNA band, if present, corresponds to the number of (CGG)ₙ/(CCG)ₙ repeats in FRAXG. The approximate number of repeats can be determined by comparing the size of the amplified band with DNA fragments of known sizes (i.e., markers). This is conveniently done using agarose gel electrophoresis. As discussed above, the absence of PCR products corresponding to a FRAXG allele generally indicates the presence of an elevated number of (CGG)ₙ/(CCG)ₙ repeats within that allele.

In the second step of the preferred embodiment, a sample of DNA from the subject is subjected to digestion by one or more restriction endonucleases, than analyzed by Southern blotting using a nucleotide probe able to hybridize to FRAXG or a restriction fragment within the digested DNA containing all or part of FRAXG. The size of the hybridizing fragment is indicative of the number of (CGG)ₙ/(CCG)ₙ repeats within FRAXG.

Restriction endonucleases used to digest the DNA obtained from the subject are chosen such that cleavage with the endonucleases produces one or more fragments containing all or part of FRAXG. The one or more fragments produced are such that the size of the fragments correlates with the number of (CGG)ₙ/(CCG)ₙ repeats within FRAXG. One or more restriction endonucleases can be used to cleave the DNA. Preferably, at least one of the restriction endonucleases chosen cleaves in a region of the genomic DNA that is outside of FRAXG (i.e., cleaves in a region flanking the (CGG)ₙ/(CCG)ₙ repeats.

Selection of the one or more restriction endonucleases to use will generally be made based on knowledge of the nucleotide sequence of the genomic regions flanking FRAXG. The nucleotide sequence of at least part of these flanking regions is known (see FIGS. 10 and 14). Normally, the nucleotide sequences of the flanking regions is analyzed by computer software that looks for restriction endonuclease recognition sites for a wide variety of restriction endonucleases within the flanking regions and identifies such sites. After such an analysis, one would preferably identify a restriction endonuclease that has a recognition site on either side of FRAXG. Preferably, cleavage of the DNA with such an endonucleases produces a fragment containing FRAXG that is between approximately 100 base pairs (bp) and 50 kilobase pairs (kbp) in size. Examples of some such endonucleases are EcoRI and HindIII. Alternatively, one would identify two different restriction endonucleases, each with a recognition site on either side of FRAXG such that cleave of the DNA with the two enzymes produces a fragment containing FRAXG that is between approximately 100 base pairs (bp) and 50 kilobase pairs (kbp) in size. Examples of pairs of some such endonucleases that can be used in combination are EcoRI and NotI, EcoRI and HindIII, NotI and HindIII, and NotI and EcoRV. Less preferably, one or two restriction endonucleases can be selected such that there is at least one recognition site within FRAXG, as long as there is at least one fragment resulting that varies in size dependent on the number of (CGG)ₙ/(CCG)ₙ repeats within FRAXG.

Southern Blot Analysis to Determine (CGG)ₙ/(CCG)ₙ Number

Once the appropriate restriction endonucleases are determined and the DNA has been cleaved with the enzymes, the cleaved DNA is separated by size, preferably using agarose gel electrophoresis. The separated DNA is then transferred from the gel to a solid support, such as a membrane. Such membranes include, but are not limited to, nitrocellulose and nylon.

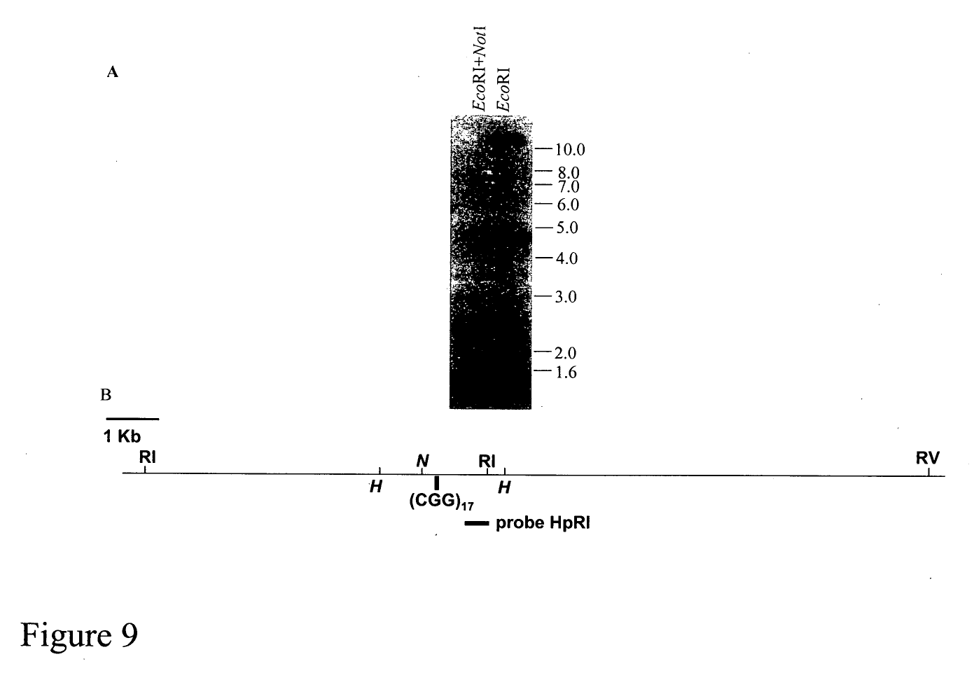

Hybridization of a nucleotide probe to the separated DNA fragments on the membrane is then performed. The nucleotide sequence of the hybridization probe is chosen so as to hybridize to the particular DNA fragment within the digested DNA that contains all of FRAXG or that contains a part of FRAXG such that the size of the fragment varies depending on the number of (CGG)ₙ/(CCG)ₙ repeats within FRAXG. The hybridization probe, therefore, is a nucleotide sequence complementary to a part of FRAXG, or to a genomic region adjacent to FRAXG, as long as that region is complementary to one strand of the DNA located within the boundaries of the fragment containing all or part of FRAXG whose size varies dependent on the number of (CGG)ₙ/(CCG)ₙ repeats within FRAXG. The probe is preferably at least 20 nucleotides in length. In one embodiment, the probe comprises a sequence having multiple CGG repeats, (CGG)₇, for example. In another embodiment, the probe comprises one or both strands of the 770 bp HpaI-EcoRI fragment shown in FIG. 9 (i.e., the HpRI probe). Many other probes can be used.

The selected nucleotide probe is then labeled and hybridized to the separated DNA fragments on the membrane. A common label for the probe is radioactive phosphorus (³²P) which is often part of a nucleoside triphosphate that is incorporated into the DNA using an enzymatic reaction, such as nick translation, random primed labeling or end labeling. Hybridization of the labeled probe to the fragment on the membrane is preferably performed under stringent hybridization conditions (i.e., conditions that do not allow mismatches during hybridization). Stringent conditions generally occur within a range from about Tₘ-5 (5° below the melting temperature of the probe) to about 20° C. below Tₘ. As used herein “highly stringent” conditions employ at least 0.2×SSC buffer and at least 65° C. As recognized in the art, stringency conditions can be attained by varying a number of factors such as the length and nature, i.e., DNA or RNA, of the probe; the length and nature of the target sequence, the concentration of the salts and other components, such as formamide, dextran sulfate, and polyethylene glycol, of the hybridization solution. All of these factors may be varied to generate conditions of stringency which are equivalent to the conditions listed above. Hybridization of the labeled probe to DNA fragments on the membrane is commonly detected using autoradiography. Other common methods for labeling DNA probes and detecting their hybridization includes, but is not limited to, non-radioactive methods, such as for example, chemiluminescent methods.

After completion of the Southern blot, the size of the hybridizing fragment, which contains all or part of FRAXG, is determined. The position of the fragment on the autoradiograph corresponds to the position of the fragment in the agarose gel (migration through the gel depends on fragment size) before transfer to the membrane support. The size of the hybridizing fragment is generally determined based on its position on the membrane relative to one or more marker DNA fragments which were run on the same agarose gel and transferred simultaneously with the DNA which had been cleaved with the restriction endonucleases. The size of the hybridizing fragment is dependent on the number of (CGG)ₙ/(CCG)ₙ repeats within the fragment and, therefore, within FRAXG.

In addition to the methods described above used in the two-step process for determining the number of (CGG)ₙ/(CCG)ₙ nucleotide triplets within FRAXG, other methods can be used. In some instances it may be possible to use the method of only one of the steps described above to determine the number of triplet repeats within FRAXG. For example, the Southern blotting method can be used alone to determine the sizes of the two FRAXG alleles. In other instances, PCR and/or Southern blotting may be combined with additional methods, such as DNA sequencing, to determine the number of triplet repeats within FRAXG.

Fiber FISH Analysis to Determine (CGG)ₙ/(CCG)ₙ Number

One additional method that can be used to determine the number of (CGG)ₙ/(CCG)ₙ nucleotide triplets within FRAXG is “fiber FISH.” One reference describing fiber FISH is Rosenberg, C., et al. 1995, High resolution DNA fiber-fish on yeast artificial chromosomes: direct visualization of DNA replication, Nat. Genet. 10(4):477-479. Fiber FISH is fluorescent in situ hybridization (FISH) that is performed on stretched or spread genomic DNA, as opposed to conventional FISH that is performed on interphase genomic DNA. In the fiber FISH method, the DNA to which the probe is hybridized is physically stretched such that the DNA is immobilized on a hybridization support (e.g., slide) as a linear DNA fiber. Because the immobilized genomic DNA is linear, when the probes hybridize to the genomic DNA, the size or length of the measured hybridization signal is related to the actual length of the genomic DNA to which the probe hybridizes. This makes it possible to relate the sizes of two different genomic DNA regions to which FISH probes hybridize.

One example of application of fiber FISH to determination of the number of (CGG)ₙ/(CCG)ₙ nucleotide triplets within FRAXG is as follows. A BAC of known length, which hybridizes to a genomic region adjacent to FRAXG, is used as a FISH probe and is hybridized to stretched genomic DNA containing FRAXG on a slide. Additionally, a probe for FRAXG, such as (CCG)₁₇, is used as a FISH probe and is hybridized to the same stretched genomic DNA on the slide. After hybridization of the two probes is complete, physical measurements are made of the lengths along the stretched genomic DNA to which each probe has hybridized (i.e., by tracing the length of the hybridization signal). Because the actual length of the BAC is known, a ratio of its actual length to the measured length of its hybridization signal can be obtained. This ratio is then used to calculate the actual length of FRAXG using the measured length of the hybridization signal from (CCG)₁₇. The calculated actual length of FRAXG is then used to determine the number of (CGG)ₙ/(CCG)ₙ nucleotide triplets therein.

In still other instances, it may be possible to use other methods known in the art to determine the number of (CGG)ₙ/(CCG)ₙ nucleotide triplets within FRAXG.

Methods for Determining Hypermethylation of Cytosines within the CpG Island Encompassing FRAXG

As shown in FIG. 15A, FRAXG is located within and is part of a CpG island. The estimated size of this CpG island is between 1.2 to 2.0 kilobase pairs in length. Hypermethylation of one or more cytosine nucleotides that are part of CpG dinucleotides within this CpG island indicates that the individual from whom the DNA was obtained is predisposed to develop symptoms of short stature.

There are a variety of methods that can be used to detect hypermethylation of a genomic region in DNA from cells of an individual. Generally, the methods used do not examine each cytosine nucleotide within the CpG island to determine its methylation state. Generally, the methods examine a few or even a single cytosine nucleotide within the CpG island. Often, hypermethylation of a few or even a single cytosine nucleotide is indicative that other cytosines within the CpG island are also hypermethylated.

Detection of CpG Methylation Using Specific Restriction Endonucleases

In one method for determining hypermethylation, a restriction endonuclease is selected that has a cytosine that is part of a CpG dinucleotide that is part of its recognition sequence. Further, the ability of the restriction endonuclease to cleave DNA at the recognition sequence is determined based on whether one or more cytosines within the recognition sequence is methylated to 5-methylcytosine. For some of these endonucleases, for example, the endonuclease will cleave the DNA at its recognition sequence if one or more cytosines within the recognition sequence are not methylated, but will not cleave if one or more cytosines is methylated. Such an endonuclease is called methylation-sensitive. For some other of these endonucleases, the endonuclease will cleave its recognition sequence if one or more cytosines within the recognition sequence are methylated, but will not cleave if there is no methylation. Such an endonuclease is called methylation-dependent. Different restriction endonucleases also exist that recognize the same recognition sequence but have differential ability to cleave the DNA at the recognition sequence based on methylation, or lack thereof, of one or more cytosine nucleotides within the recognition sequence.

Such restriction endonucleases are used when one or more cleavage recognition sites for the endonuclease are present within the CpG island that contains FRAXG. The endonuclease is used to cleave the DNA from cells of an individual and it is determined whether the recognition sites within the CpG island were actually cleaved. Knowledge of the ability of the particular endonuclease to cleave the sequence based on its methylation pattern is used to determine if cleavage, or lack thereof, of the recognition site within the CpG island indicates that one or more cytosines are methylated or not. Often, DNA from another individual, where the methylation status of the particular cytosines within the recognition site is known, is used as a control.

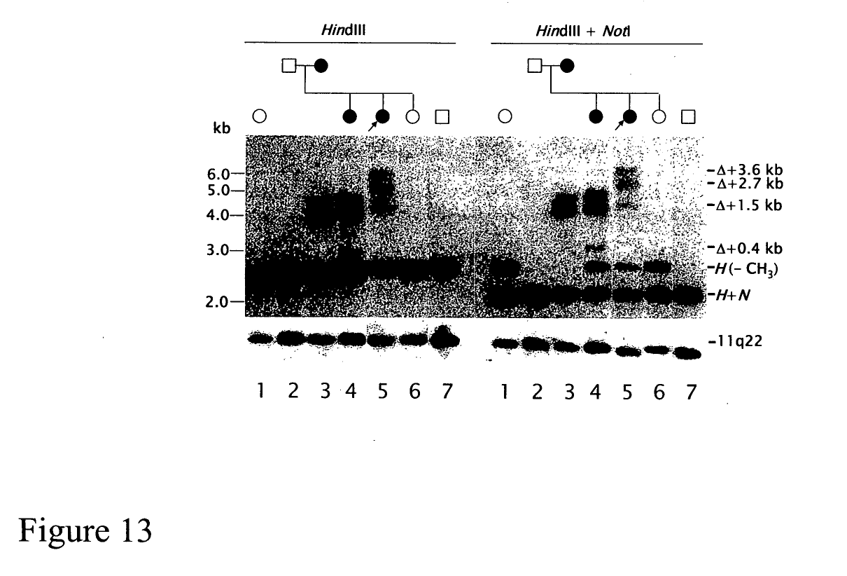

Such restriction endonucleases are generally used within the context of a technique that can be used to detect and/or display DNA fragments. One such technique is Southern blotting. FIG. 13 and its discussion above demonstrate use of one restriction endonuclease, NotI, whose ability to cleave DNA is methylation-dependent, in Southern blotting to detect hypermethylation within the CpG island containing FRAXG.

Such methylation-sensitive or methylation-dependent restriction endonucleases can be combined with still other techniques to determine hypermethylation. In one method, PCR primers are chosen to amplify a genome region within the CpG island containing FRAXG. The genome region to be amplified also contains one or more cleavage recognition sites for methylation-sensitive or methylation-resistant restriction endonucleases. DNA isolated from cells of an individual is treated with the particular restriction endonuclease before the DNA is used as a template in the PCR reaction. If the particular cleavage recognition site within the region to be amplified is cleaved, the PCR reaction will not successfully amplify the template. If the particular cleavage recognition sites within the region to be amplified is not cleaved, the PCR reaction will amplify the template. Knowledge of when the particular endonuclease cleaves the DNA combined with the presence or absence of a PCR amplification product is used to determine whether there is methylated cytosine within the particular cleavage recognition site within the CpG island.

In one particular embodiment of this method, at least one PCR primer is made to hybridize to a region of the CpG island that contains CpG dinucleotides and within which a methylation-sensitive restriction endonuclease recognition site is present. The DNA from the individual is treated with the endonuclease before being used in PCR. In the case where there is no methylation, the restriction endonuclease cleaves the DNA and the PCR primer designed to hybridize to the sequence does not hybridize since the sequence has been cleaved by the restriction endonuclease. The PCR reaction does result in amplification of a fragment in this case. In the case where there is methylation, the restriction endonuclease does not cleave the DNA and the PCR primer designed to hybridize to the sequence does hybridize since the DNA has not been cleaved by the restriction endonuclease. The PCR reaction will result in amplification of the fragment.

Detection of CpG Methylation Using Bisulfite Treatment

Other methods, not necessarily using restriction endonucleases, to detect methylation and determine hypermethylation, can be used. In one method bisulfite treatment of the genome DNA isolated from an individual is used to change the methylated cytosines therein to a different nucleotide base. Sodium bisulfite converts unmethylated cytosine to uracil. If the DNA contains cytosine and is treated with sodium bisulfite, the treated DNA will contain uracil in place of the cytosine nucleotides. If the DNA contains 5-methylcytosine and is treated with sodium bisulfite, the treatment will not change the DNA. The 5-methylcytosine nucleotides will still be 5-methylcytosines. The conversion of cytosine to uracil, in the first case, is then detected using various techniques, methylation-sensitive PCR (discussed below) being one of these techniques. Other detection methods use various DNA sequencing techniques. One such technique is genomic sequencing. Other methods are known and can be used.

“Methylation-sensitive PCR” (MSP) refers to a PCR in which amplification of the template DNA which has been treated with sodium bisulfite is attempted. Two sets of primers are designed for use in MSP. Each set of primers comprises a forward primer and a reverse primer. One set of primers, called methylation-specific primers, will amplify the bisulfite-treated DNA template sequence if cytosine nucleotides in CpG dinucleotides within the CpG island are methylated. Another set of primers, called unmethylation-specific primers, will amplify the bisulfite-treated DNA template if cytosine nucleotides in CpG dinucleotides within the CpG island are not methylated.

Each primer set comprises a forward and reverse primer, as discussed earlier. Selection of such primers depends on one of the two primers in each pair having a sequence complementary to a DNA sequence (a target sequence) within the CpG island. The sequences of the methylation-specific and unmethylation-specific primers are different since hybridization of the primers is to a sequence containing a cytosine or uracil, depending on whether the cytosines were methylated.

Two separate PCR reactions are then run. Both reactions use the bisulfite-treated genomic DNA. In one of the reactions, methylation-specific primers are used. In the case where cytosine within CpG dinucleotides of the target sequence of the DNA are methylated, the methylation-specific primers will amplify the bisulfite-treated template sequence in the presence of a polymerase and an MSP product will be produced. If cytosine within CpG dinucleotides of the target sequence of the DNA are not methylated, the methylation-specific primers will not amplify the bisulfite-treated template sequence in the presence of a polymerase and an MSP product will not be produced.

In the other reaction, unmethylation-specific primers are used. In the case where cytosine within CpG dinucleotides of the target sequence of the DNA are unmethylated, the unmethylation specific primers will amplify the bisulfite-treated template sequence in the presence of a polymerase and an MSP product will be produced. If cytosine within CpG dinucleotides of the target sequence of the DNA are methylated, the unmethylation-specific primers will not amplify the compound-converted template sequence in the presence of a polymerase and an MSP product will not be produced.

Other methods known in the art can be used to determine hypermethylation of cytosine nucleotides that are part of CpG dinucleotides within the CpG island containing FRAXG.

### EXAMPLES

Further details of the invention can be found in the following examples, which further define the scope of the invention.

### Example 1

### Identification Mapping and Characterization of FRAXG: Case Study of a Finnish Family

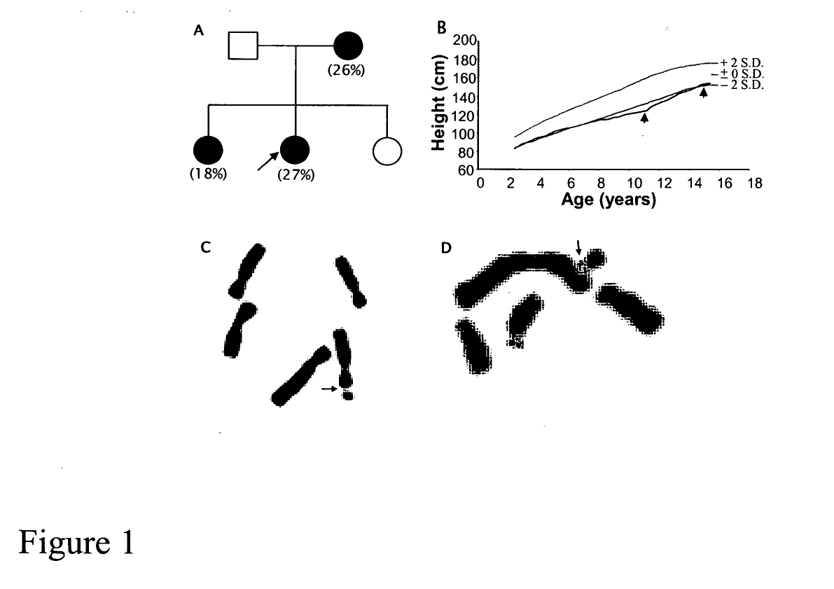

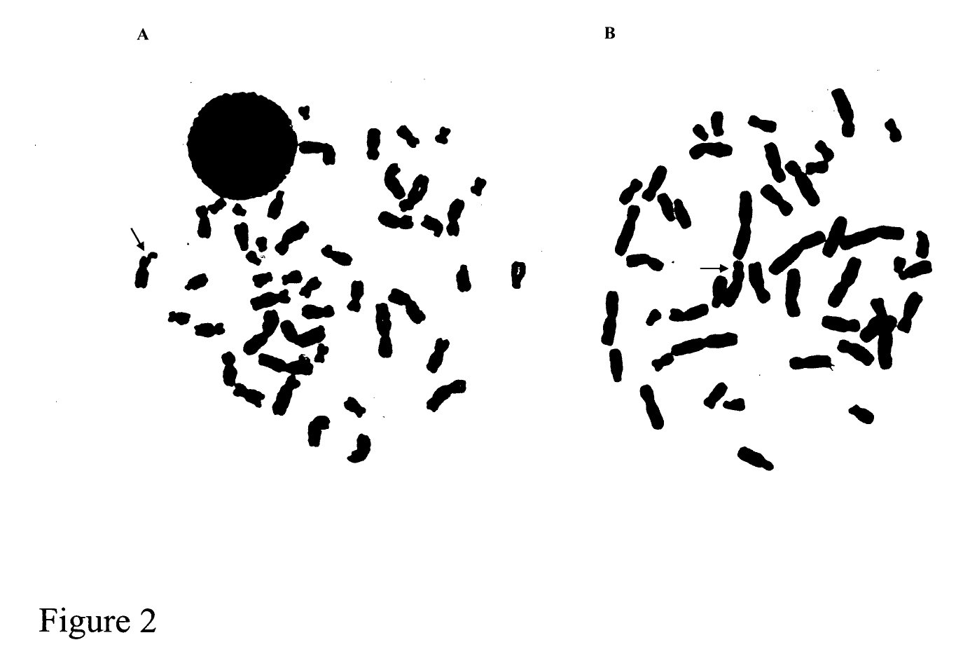

The proband (i.e., the initial subject in a family to present a disorder who causes initiation of a genetic study on the family) was a Finnish girl of seven years old when she was brought to a physician's attention due to her short stature. At age 9.4 years, her weight was 21.6 kg and her height was 118.3 cm (3.2 standard deviations below the mean for that age). No other complaints were mentioned. No abnormal eating or sleeping habits were mentioned. No chronic fever, diarrhea, or chronic pain was complained of. The girl was delivered naturally without any incidents at 41 weeks of gestation. Her body weight and height (47 cm) were within normal range at birth. She was the second child of nonconsanguineous parents (FIG. 1A). Physical examinations were generally normal except her height. Her height was below the fifth percentile of her peers. The ratio of her upper body length over her lower limb length was normal. No physical dysmorphia was identified. Her intelligence and speech were normal. Hair and skin were normal. No brittle hair and no abnormal skin temperature were observed. Her external sex organ was normal. Body temperature, heart rate, and blood pressure were normal. Regular laboratory tests including serum sodium, potassium, chloride, and calcium were normal. She had two sisters. Both were normal with normal height. Her parents were normal with normal height. The initial diagnosis of her condition was idiopathic short stature. The girl had normal endocrinological findings. From age 11.2 to 14.8 years, she was given growth hormone treatment and had a positive response. At 15.4 years old her height was 155 cm. FIG. 2B shows her growth curve together with the growth curves for normal Finnish girls.

### Example 2

### Identification of FRAXG, a Folate-Sensitive Fragile Site Located Close to the Border of Xp21 and Xp22 in the Finnish Family

This study was performed when a chromosome study of the proband was requested at age 9.4 years due to her unexplained short stature. Peripheral blood was drawn from the proband and other family members using standard techniques. Induction of FRAXG was carried out following the recommended procedures for the induction of rare, folate-sensitive fragile site (Jacky, P. B., Ahuja, Y. R., et al., 1991, Guidelines for the preparation and analysis of the fragile X chromosome in lymphocytes, Am J Med Genet 38(2-3):400-3). Briefly, cells present in the peripheral blood were cultured for four days after standard treatment as described by Verma and Babu, 1989, Human Chromosomes: Manual of Basic Techniques, p. 240. Metaphase spreads were prepared by standard techniques and stained by either Giemsa for solid staining or Trypsin-Giemsa for banding.

The results showed that the karyotype of the proband was normal, 46, XX, but in three metaphases out of 49 studied a fragile site was observed at the border of Xp21 and Xp22 (FIG. 1C), which indicated the presence of a novel fragile site in this region in the proband. Trypsin-Giemsa banding further confirmed the location of this novel fragile site (FIG. 1D). Subsequent induction studies (i.e., showing enhanced expression under culture condition for “rare heritable, folate-sensitive fragile site” or RHFF) confirmed that the novel fragile site is a rare, folate-sensitive fragile site with expression frequency of around 27%. Similar studies were carried out on the proband's parents and two sisters. Similar fragile sites were identified at the same locations on chromosomes from the proband's mother and elder sister with frequencies of 26% and 18% respectively (FIG. 1A). No similar fragile sites were identified from the proband's father or younger sister. Thus, a novel rare heritable folate-sensitive fragile site located close to the border of Xp21 and Xp22, named FRAXG following standard nomenclature was identified from the proband with short stature.

### Example 3

### Induction of FRAXG from the Proband's Lymphoblastoid Cell Line

Lymphoblastoid cell lines (LBCL) from all family members were established from peripheral lymphocytes. Two inducing conditions for RHFFS in LBCL were used. One is medium 199 (Gibco BRL) plus FudR at concentration of 10⁻⁶, 5×10⁻⁷ or 10⁻⁷ M for 24 or 48 hours. Another is medium 199 plus MTX at concentration of 10⁻⁷ M for 24 or 48 hours. As shown in FIG. 2, FRAXG was observed as both a chromatid break (Panel A) and non-staining gap (Panel B). Under the inducing conditions tested, medium 199 plus 10⁻⁷ M FudR gave the highest induction rate of FRAXG (5-7%). Compared to PBL, this is about 25% of that from fresh PBL. Successful induction of FRAXG in LBCLs not only confirmed the expression of this novel fragile site in the Finnish kindred, but also provided sufficient samples for the subsequent fine fluorescence in situ hybridization (FISH) mapping of FRAXG.

### Example 4

### Localization of FRAXG to a Region of Xp22.1 Using FISH Analysis

The chromosomal region containing FRAXG was determined by fluorescence in situ hybridization (FISH) using mapped clones, such as YACs (yeast artificial chromosomes) or BACs (bacterial artificial chromosomes), as probes. The YACs and BACs were not from the kindred shown in FIG. 1A. Rather, the YAC and BAC DNAs were from normal individuals, and therefore, contained “normal” DNA (i.e., did not contain number of (CGG)ₙ/(CCG)ₙ triplets at FRAXG significantly greater in number than the average number of repeats in a population of normal individuals).

Based on the GTG banding of metaphase chromosomes expressing FRAXG, FRAXG was tentatively mapped to Xp22.1 (FIGS. 1C and 1D). To further fine map FRAXG, a contig of six YACs from Xp22.1 was used in FISH to determine the location of FRAXG. The clones are described in two references (Alitalo, T., Francis, F., Kere, J., Lehrach, H., Schlessinger, D. and Willard, H. F., 1995, A 6-Mb YAC contig in Xp22.1-p22.2 spanning the DXS69E, XE59, GLRA2, PIGA, GRPR, CALB3, and PHKA2 genes, Genomics 25:691-700; Ferrero, G. B., Franco, B., Roth, E. J., Firulli, B. A., Borsani, G., Delmas-Mata, J., Weissenbach, J., Halley, G., Schlessinger, D., Chinault, A. C., et al., 1995, An integrated physical and genetic map of a 35 Mb region on chromosome Xp22.3-Xp21.3, Hum Mol Genet 4:1821-1827). Clones y911G5, y827E10, y946F5, and y811D11 were purchased from Research Genetics (Huntsville, Ala.). Clones y295D1 and y517G4 were obtained from CEPH (France).

YAC DNA was isolated from host cells using standard methods. An inter-Alu PCR was used to amplify the YAC inserts with the combinations of primers Alu1 (5′-GGATTACAGGYRTGAGCCA-3′; SEQ ID NO. 6) and Alu2 (5′-RCCAYTGCACTCCAGCCTG-3′; SEQ ID NO. 7) using procedures previously described (Liu, P., Siciliano, J., Seong, D., Craig, J., Zhao, Y., de Jong, P. J. and Siciliano, M. J., 1993, Dual Alu polymerase chain reaction primers and conditions for isolation of human chromosome painting probes from hybrid cells, Cancer Genet Cytogenet 65:93-99). Five of the YACs were individually labeled by FITC-dUTP and used in FISH. The procedures for FISH were as described (Kievits, T., Dauwerse, J. G., Wiegant, J., Devilee, P., Breuning, M. H., Cornelisse, C. J., van Ommen, G. J. and Pearson, P. L., 1990, Rapid subchromosomal localization of cosmids by nonradioactive in situ hybridization, Cytogenet Cell Genet 53:134-136).

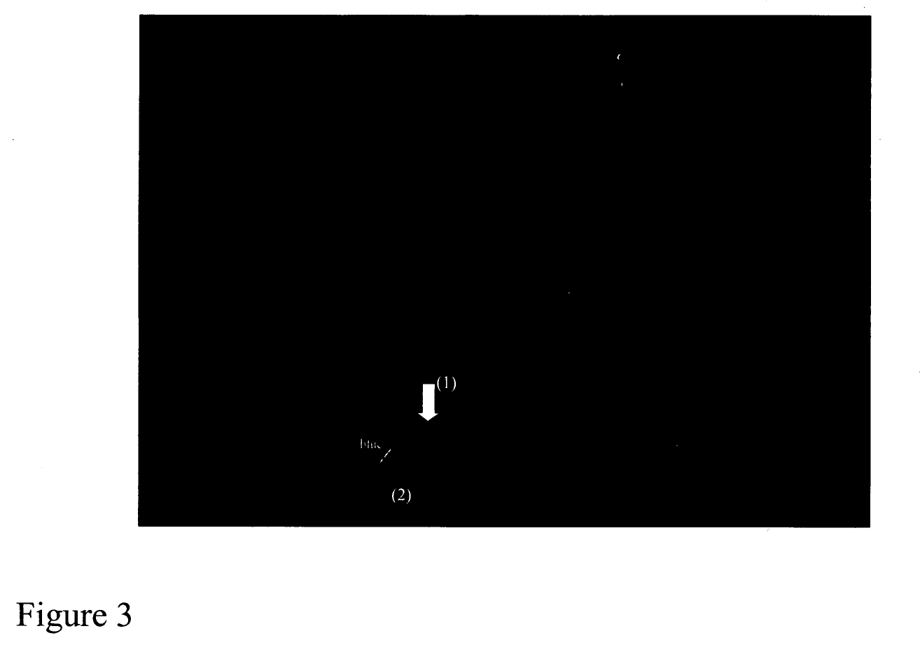

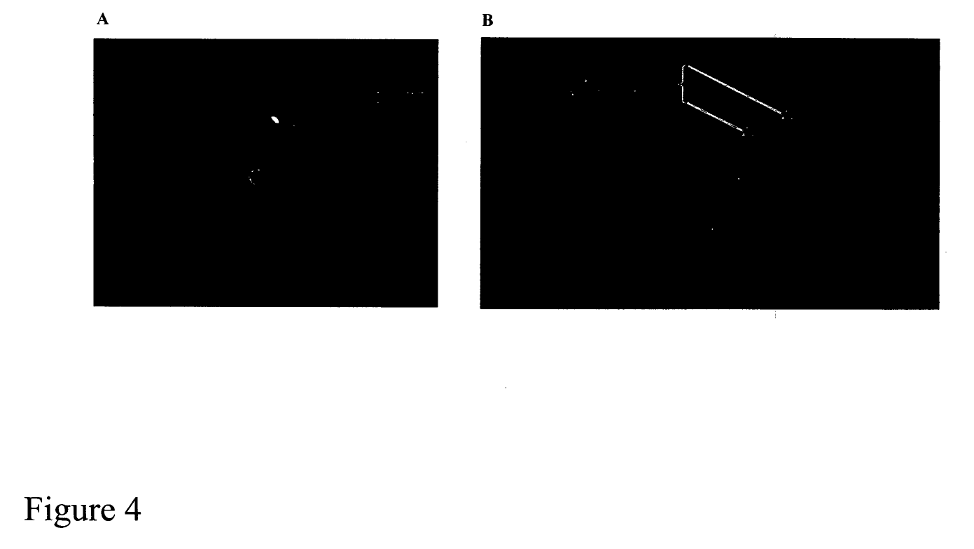

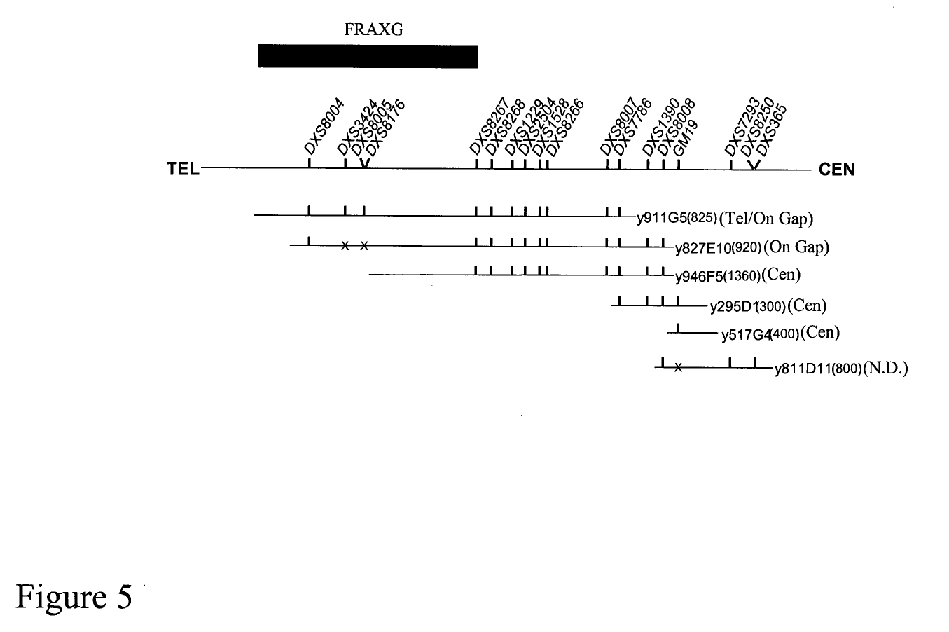

To determine the relative location of a YAC to FRAXG, at least five metaphase spreads expressing FRAXG and a good FISH signal from the respective YACs were identified and captured. Each signal was designated as centromeric when the signal was centromeric to FRAXG; telomeric when the signal was located telomeric to FRAXG; and on gap when the signal and FRAXG were located in the same position. The position of a YAC to FRAXG was determined based on the location of the majority of FISH signals relative to FRAXG. As shown in FIG. 3, y827E10 was located right onto the broken chromatids (see indicated arrow in figure). The red signal (arrow labeled “red) was from b733018, which has been mapped to Xp22.31. It was used to identify the telomeric part of Xp—either still attached or broken off. An X-chromosome centromere specific probe, CEPX alpha (Vysis), was also included to identify the X chromosome. Shown in FIGS. 4A and 4B are two representative FISH images showing y911G5 and y946F5 located telomeric and centromeric to FRAXG, respectively. FIG. 5 summarizes the FISH results of the locations of the five YACs relative to FRAXG. Based on these mapping data, FRAXG is located in a region in Xp22.1, which is covered telomerically by y911G5 and centromerically by y946F5, a region of about 1 Mb (indicated by the solid bar in FIG. 5). The FISH mapping with the YAC contig defined the region of FRAXG.

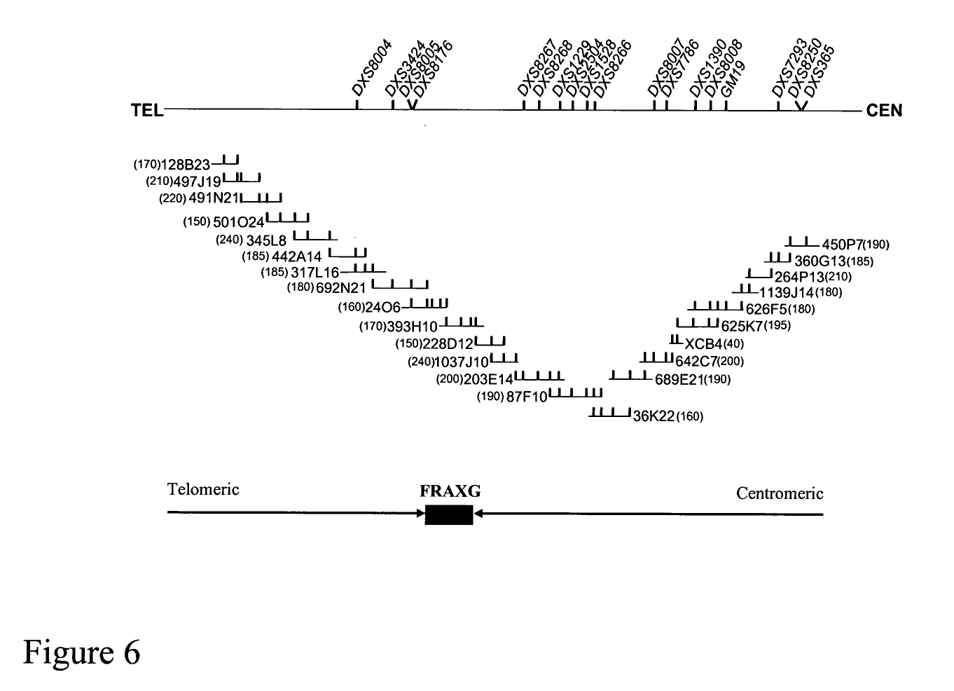

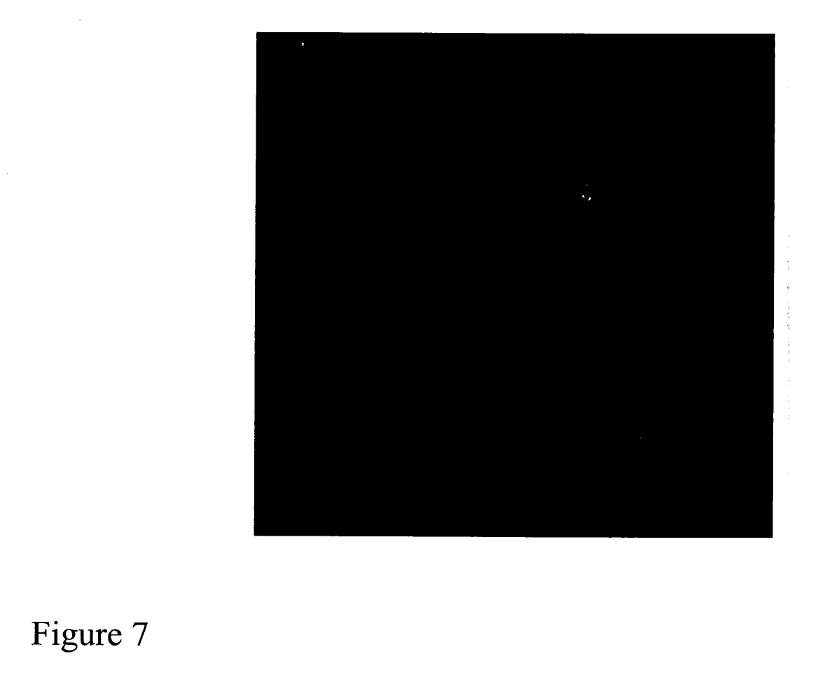

As YACs on average have inserts of hundreds of kb, other clones that have smaller inserts, were used to further fine map FRAXG. BACs were chosen as they are highly stable and less chimeric. They have on average insert size of 150 kb-250 kb. BAC DNA can also be directly sequenced. During the course of this project, a complete BAC-cosmid contig was assembled to cover the region covered by the YAC contig (Zhang, S and Krahe, R., 2002, Physical and transcript map of a 2-Mb region in Xp22.1 containing candidate genes for X-linked mental retardation and short statute, Genomics 79:274-275). A total of 23 BACs covers this region with minimal overlapping. These BACs were used in the FISH mapping of FRAXG (as described above using YAC clones). As summarized in FIG. 6, all BACs centromeric to b228D12, including b228D12, were centromeric to FRAXG, while all BACs telomeric to b692N21, including b692N21, were telomeric to FRAXG. Therefore, FRAXG was mapped to a region of less than 200 kb covered by two overlapping BACs, b393H10 and b2406. As shown in FIG. 7, b393H10 is located right on the unstaining gap of FRAXG, which indicates that b393H10 contains the region of FRAXG.

### Example 5

### Identification and Characterization of (CGG)ₙ/(CCG)ₙ Trinucleotide Repeats in BACs

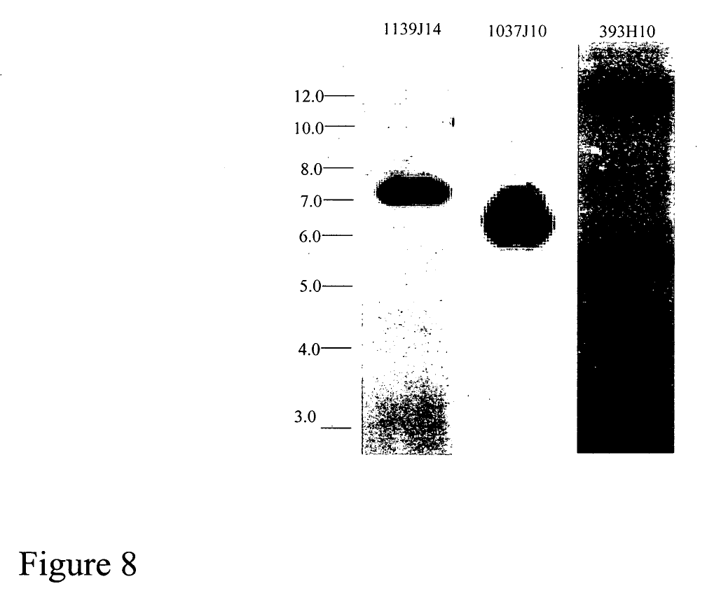

The 23 BACs comprising the minimal tiling path of the region were digested with EcoRI and investigated by Southern analysis for the presence of CGG/CCG trinucleotide repeats with a radiolabeled (CCG)₇ probe. As shown in FIG. 8, three distinct (CGG)ₙ/(CCG)ₙ-positive fragments from b1139J14, b1037J10, and b393H10 were detected. As shown in FIG. 6, 1139J14 and 1037J10 map centromeric to FRAXG. Only 393H10 lies in the FRAXG candidate region as defined by the FISH analysis. Therefore, further mapping of the (CGG)ₙ/(CCG)ₙ repeats within 393H10 was performed.

The (CGG)ₙ/(CCG)ₙ-containing fragment within 393H10 was further mapped to a 1.6 kb EcoRI-NotI fragment (FIG. 9). After it was cloned into the EcoRI-NotI site of the pZero2 vector, the whole 1.6 kb fragment was sequenced. A run of seventeen consecutive CGG triplets was identified in this particular allele. The complementary strand contained CCG triplets. This sequence is designated as (CCG)₁₇. Part of this sequence is shown in FIG. 10 (SEQ ID NO.1) with the (CCG)₁₇ repeat in bold. As illustrated in FIG. 9, Panel B, the (CCG)₁₇ is located 261 bp downstream of the NotI site. A 770 bp HpaI-EcoRI fragment from this region (designated HpRI), which does not contain the (CCG)₁₇, was used in subsequent Southern blot analyses. BLAST sequence analysis identified no known homologous sequences. Further sequence analysis indicated that this repeat is within a CpG island. Therefore, the CpG island encompasses FRAXG. The size of the CpG island was estimated to be between 1 to 2 kilobase pairs in length.

The above studies mapped FRAXG to the human genome and provided the DNA sequence of FRAXG and the surrounding region. These studies showed 17 (CGG)ₙ/(CCG)ₙ trinucleotides in the DNA from the 393H₁₀BAC, which is DNA from a normal individual. A study was done to determine the distribution of the number of CCG trinucleotides at this locus in normal Finnish individuals.

To estimate the copy number of (CGG)ₙ/(CCG)ₙ trinucleotide repeats in a normal population, a group of 286 random-selected normal Finnish males were studied by polymerase chain reaction (PCR). Fluorescence dye-labeled oligonucleotides 393H10_F: FAM-GTGGGAGGCGGCGGCAGAGTGAGG (SEQ ID NO. 2), and 393H10_R: GCCCCATCCGCCACCCCGAGAACC (SEQ ID NO. 3) were derived from the sequences flanking the (CGG)₁₇ repeat (FIG. 10), and were used as primers to amplify genomic DNA using PCR with standard techniques. The copy number of the (CGG)ₙ/(CCG)ₙ repeat in each product was estimated by comparing it with a sequence containing known numbers of the (CGG)ₙ/(CCG)ₙ repeats. FIG. 11 summarizes the results. The Finnish population contained nine to 21 copies of (CGG)ₙ/(CCG)ₙ triplets at FRAXG loci. Almost half of the population studied had 13 copies of (CGG)ₙ/(CCG)ₙ triplets. More than 85% of the population contained 12-16 copies of the (CGG)ₙ/(CCG)ₙ triplets.

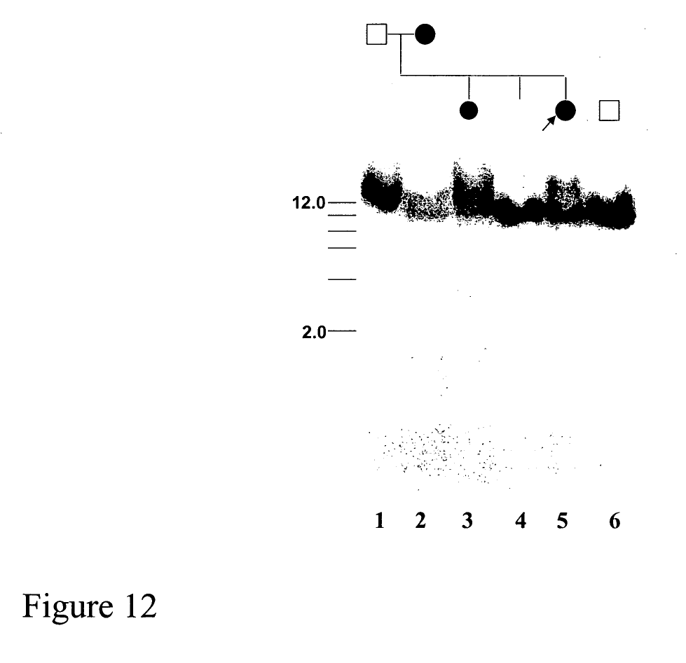

To determine the number of (CGG)ₙ/(CCG)ₙ triplets in members of the kindred shown in FIG. 1A, Southern blots were performed. FIG. 12 is an EcoRI-digested genomic DNA Southern blot of genome DNA isolated from members of the kindred shown in FIG. 1A, hybridized with the radiolabeled 770 bp HpaI-EcoRI fragment (HpRI) (FIG. 9). In lanes 2, 3, and 5, a higher molecular weight fragment in addition to the common fragment was detected. Samples in these three lanes were from the proband's mother (Lane 2), sister (Lane 3) and the proband (Lane 5), respectively. All three had been shown to express FRAXG (i.e., have amplification of the nucleotide triplets). The proband's father and another sister did not express FRAXG, and no second band was detected. A single, approximately 12 kb EcoRI fragment was detected in a normal male control (lane 6). Genotyping of the X chromosomes in this family with 11 X-chromosome microsatellite markers indicated that the three X chromosomes with the expansion were the same chromosomes inherited.

### Example 6

### Determination of Numbers of (CGG)ₙ/(CCG)ₙ Repeats and of Hypermethylation

To determine whether expansion of the triplet affected the methylation of the CpG island that encompasses FRAXG (see FIG. 15A for approximate location of the CpG island), additional Southern blots were performed. In FIG. 13, a methylation-sensitive restriction enzyme, NotI, was included in the genomic DNA Southern blot. When the two Cs in the GCGGCCGC sequence of NotI site are methylated, the NotI cleavage at this site is blocked. As shown in FIG. 13, in the HindIII single digest, a common 2.6 kb fragment was present in all individuals. The expanded fragments and smears were detected in the FRAXG-expressing individuals. As shown in lanes 3, 4, and 6 in the left half, the expansion was further expanded as the maternal X chromosome was passed to her daughters, which suggested the germline instability of the expansion. The largest expansion was observed in the proband. There are three major expanded fragments together with the smear in the proband: by +1.5 kb, +2.7 kb, and +3.6 kb. The calculated increase of the copy number of (CGG)ₙ/(CCG)ₙ is approximately 500, 900, and 1200. In the HindIII and NotI double digestion in the right half in FIG. 13, lane 1 is a non-related normal female. Half of her X chromosome DNA was methylated due to the random X chromosome inactivation, as indicated by about equal amounts of 2.6 kb HindIII and 2.0 kb HindIII and NotI fragments. Lane 7 is a normal male control. All his X chromosome is unmethylated, as indicated by no remaining 2.6 kb HindIII fragment after the HindIII and NotI double digestion. In lanes 3, 4, and 6, the NotI sites in all the expanded fragments were methylated as indicated by the same amount of remaining fragments compared with those in the HindIII single digestion. Lane 5 is the normal sister of the proband. The methylation pattern is the same as the normal female control (Lane 1), indicating the random X chromosome inactivation. Similar results were observed when other methylation-sensitive enzymes EagI, HpaII, and SacII were used. These data together indicated that the FRAXG CpG island associated with the expanded (CGG)ₙ/(CCG)ₙ in FRAXG individuals (see FIG. 15) is preferentially methylated.

### Example 7

### Transcripts and Expression Levels from the FRAXG Region

To search for genomic regions that encoded transcripts that are associated with the CpG island and the (CGG)ₙ/(CCG)ₙ repeat, regions flanking FRAXG were sequenced. First, a high density filter with EcoRV-NotI human genomic DNA plasmid pBluescript clones (courtesy of Dr. Christoph Plass) was screened by hybridization with the radiolabeled 770 bp HpaI-EcoRI fragment (HpRI). A single hybridizing clone, p68H2, was identified. This clone and the BAC clone 393H10 were sequenced. A 6882 base pair genomic sequence (GenBank AY0922821) of the region was assembled from these sequences (FIG. 14; SEQ ID NO. 4). BLAST sequence analysis against the NCBI human EST database identified a single EST, EST2660055 (GenBank accession number AA679533). EST2660055 matched to the genomic sequence 2688-2814 bp and 5150-5705 bp in the 6882-bp fragment, which indicated that it consisted of two exons. Transcription of EST2660055 in lymphoblastoid cell lines was verified by RT-PCR with primers derived from the two separate exons, and subsequent cloning and sequencing.

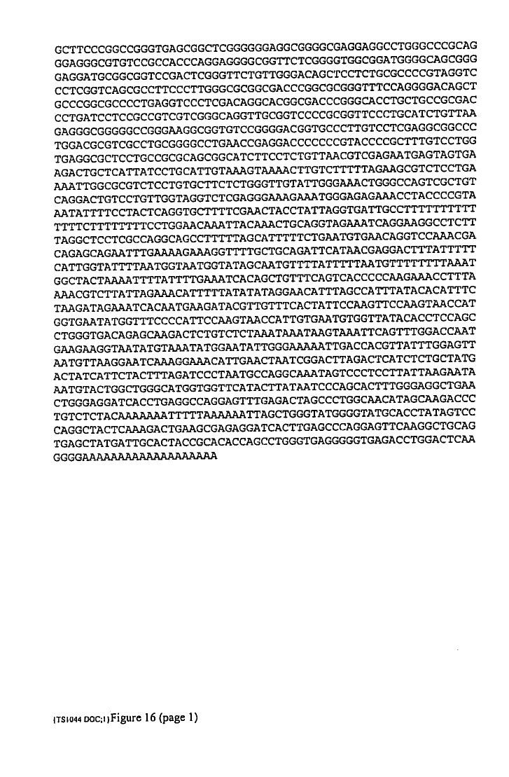

Further sequence analysis of the 6882-bp fragment using the program FirstEFprogram (Davuluri, R. V., Grosse, I and Zhang, M. O., 2001, Computational identification of promoters and first exons in the human genome, Nat. Genet. 29:412-417) revealed the presence of a putative promoter region from 901-1470 bp (FIG. 15A) and a predicted first exon from 1573-2133 bp. RT-PCR with a forward primer (1866-1886 bp) from the predicted first exon and a reverse primer (5404-5381 bp) from EST2660055 verified the transcription of the predicted first exon and showed that the predicted first exon is the direct extension of EST2660055 (FIG. 15). Thus a transcript, named FXGC for FRAXG associated gene, of 1793 bp (FIG. 16; GenBank: AY092822; SEQ ID NO. 5) with confirmed transcription of 1505 bp was isolated from the FRAXG region. BLAST sequence analysis demonstrated that FXGC shares no sequence homology to any other known genes.

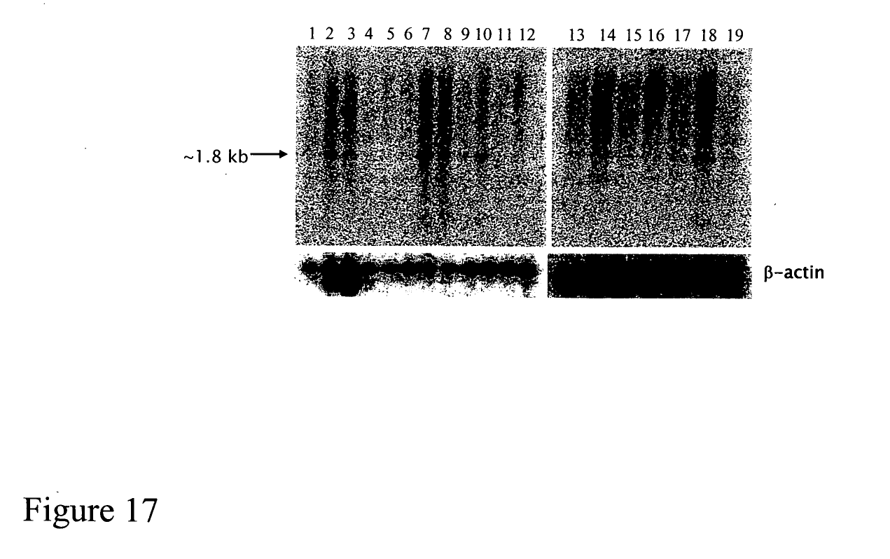

To study the expression of FXGC and to estimate the size of the endogenous FXGC transcript, human multiple tissue Northern blots were hybridized with a 429-bp probe derived from the first exon of FXGC. For Northern blot analysis of FXGC, a human multiple tissue Northern blot (Clontech) was hybridized with a 429 bp probe amplified from the first exon of FXGC using primers GIF, GGTTCTCGGGGTGGGGGATGG (SEQ ID NO. 8) and G1R, GACGTTAACAGAGGAAGATGC (SEQ ID NO. 9). As shown in FIG. 17, FXGC was transcribed mainly as a 1.8-kb fragment in almost all the tissues tested, notably heart, skeletal, kidney, liver, placenta, and bone marrow. Similar expression patterns were obtained by independent RT-PCR using cDNAs synthesized from different human tissue RNAs.

### Example 8

### Determination of (CGG)ₙ/(CCG)ₙ Triplet Repeat Number within FRAXG

Genomic DNA was extracted from blood samples of two individuals. The DNAs were used as templates in separate PCR reactions using a forward primer, (SEQ ID NO. 2), of sequence 5′-GTGGGAGGCGGCGGCAGAGTGAGG-3′ and a reverse primer, (SEQ ID NO. 3), of sequence 5′-GCCCCATCCGCCACCCCGAGAACC-3′. After the PCR reactions were completed, a portion of each reaction was analyzed using agarose gel electrophoresis. DNA size markers were also electrophoresed through the agarose gel. The PCR data for the two individuals were as follows:

The first patient showed 2 amplified bands from the PCR reaction. One band was a DNA fragment of approximately 175 base pairs (bps) in length and the other band was a DNA fragment of approximately 160 bps in length. These data indicated that both FRAXG alleles in this individual contained from approximately 20 to 30 copies of the (CGG)ₙ/(CCG)ₙ triplet repeat.

The second individual showed only a single amplified band from the PCR reaction. The single band was a DNA fragment of approximately 175 bps in length, indicating that one FRAXG allele in this individual contained approximately 20 to 30 copies of the (CGG)ₙ/(CCG)ₙ triplet repeat. The presence of only one band in the PCR reaction suggested that the other FRAXG allele contained a highly elevated number of (CGG)ₙ/(CCG)ₙ triplet repeats such that the PCR reaction was unable to amplify across the FRAXG region.

To examine the FRAXG allele in individual two, suspected to contain highly elevated numbers of triplet repeats, DNA from the individual was treated with HindIII restriction endonuclease. After the treatment, the DNA was electrophoresed through an agarose gel, then the DNA was transferred from the gel onto a nylon hybridization membrane. The DNA fragments on the membrane were hybridized under stringent conditions to a radiolabeled HpRI probe (see FIG. 9B). After hybridization, the membrane was exposed to film and an autoradiograph was obtained. The autoradiograph showed a band representing a DNA fragment of approximately 5.6 kilobase pairs (kbps). Since the genomic HindIII fragment encompassing FRAXG was approximately 2.6 kbps in size when FRAXG contained an unelevated number of (CGG)ₙ/(CCG)ₙ triplet repeats (see FIG. 9B), the presence of a 5.6 kbps band indicated that this FRAXG allele contained approximately 1000 (CGG)ₙ/(CCG)ₙ triplet repeats (1000×3 bps=3.0 kbps). The data indicated that the individual had one FRAXG allele that contained an elevated number of (CGG)ₙ/(CCG)ₙ triplet repeats.

### Example 9

### Determination of Hypermethylation of the CpG Island Containing FRAXG

DNA from the first individual in Example 1 was treated with HindIII in a first reaction and with HindIII and NotI in a second reaction. DNA from the second individual in Example 1 was treated with HindIII in a first reaction and with HindIII and NotI in a second reaction. The two digest reactions from each individual were electrophoresed through an agarose gel and the DNA transferred onto a nylon hybridization membrane, as described above. The membrane was then hybridized, under stringent conditions, with a radiolabeled probe consisting of the 0.9 kbps HindIII-NotI fragment immediately leftward of the FRAXG site (see FIG. 9B). After hybridization, the membrane was exposed to film and an autoradiograph was obtained. The data were as follows:

DNA from the first individual, that was digested with HindIII, showed a single band of approximately 2.6 kbps in size. The same DNA, digested with HindIII and NotI, showed a single band of approximately 0.9 kbps in size. As is known from the study described in Example 1, the first individual had two FRAXG alleles, both having an unelevated number of (CGG)ₙ/(CCG)ₙ triplet repeats. The decrease in size of the hybridizing band from 2.6 kbps to 0.9 kbps was due to cleavage of the DNA from both alleles at the NotI immediately leftward of FRAXG (see FIG. 9B). Cleavage at NotI occurred only when the NotI recognition sequence was not methylated.

DNA from the second individual, that was digested with HindIII showed one band of approximately 2.6 kbps in size, representing the FRAXG allele containing an unelevated number of (CGG)ₙ/(CCG)ₙ triplet repeats, and a second band of approximately 5.6 kbps in size, representing the FRAXG allele containing about 1000 (CGG)ₙ/(CCG)ₙ triplet repeats. DNA from the second individual, digested with HindIII and NotI, showed one band of approximately 0.9 kbps in size, representing cleavage at the NotI site leftward of FRAXG in the unelevated allele. A second band of approximately 5.6 kbps in size was also present. This 5.6 kbps band represented the HindIII fragment encompassing the 1000 copies of (CGG)ₙ/(CCG)ₙ in the elevated FRAXG allele. This band was present because the NotI site immediately leftward of FRAXG in the elevated allele was not cleaved due to its methylation. If this NotI site was not methylated and, therefore, was cleaved by NotI, the probe would hybridize to a 0.9 kbps fragment, not a 5.6 kbps fragment.

### Example 10

### Establishment of Lymphoblastoid Cell Lines from Kindred Individuals

Lymphoblastoid cell lines were established from peripheral blood lymphocytes according to the protocols described in Jacobs, P. A., Hunt, P. A., Mayer, M., Wang, J. C., Boss, G. R. and Erbe, R. W. (1982), Expression of the marker (X) (q28) in lymphoblastoid cell lines. Am J Hum Genet 34: 552-557, and in Abruzzo, M. A., Hunt, P. A., Mayer, M., Jacobs, P. A., Wang, J. C. and Erbe, R. W. (1986), A comparison of fragile X expression in lymphocyte and lymphoblastoid cultures. Am J Hum Genet 38: 533-539.

1. A method for identifying an individual who is predisposed to developing short stature, comprising: assaying a sample of DNA from the individual for the number of (CGG)ₙ/(CCG)ₙ nucleotide triplets in the FRAXG CpG island, wherein an increased number of (CGG)ₙ/(CCG)ₙ nucleotide triplets in at least one FRAXG allele in the individual, as compared to the average number of (CGG)ₙ/(CCG)ₙ nucleotide triplets in FRAXG alleles from a normal population of individuals, indicates the individual has an increased likelihood of developing short stature.

2. The method according to claim 1 comprising, assaying the DNA from the individual for methylation of cytosine nucleotides within the FRAXG CpG island, wherein hypermethylation of one or more of the cytosine nucleotides indicates the individual has an increased likelihood of developing short stature.

3. A method for identifying an individual who is capable of transmitting to its offspring an increased likelihood of developing short stature, comprising: assaying a sample of DNA from the individual for the number of (CGG)ₙ/(CCG)ₙ nucleotide triplets in the FRAXG CpG island, wherein an increased number of (CGG)ₙ/(CCG)ₙ nucleotide triplets in at least one FRAXG allele in the individual, as compared to the average number of (CGG)ₙ/(CCG)ₙ nucleotide triplets in FRAXG alleles from a normal population of individuals, indicates that offspring receiving from the individual a FRAXG allele having an increased number of (CGG)ₙ/(CCG)ₙ nucleotide triplets will have an increased likelihood of developing short stature.

4. The method according to claim 3 comprising, assaying the DNA from the individual for methylation of cytosine nucleotides within the FRAXG CpG island, wherein hypermethylation of one or more of the cytosine nucleotides indicates that offspring receiving from the individual a FRAXG allele having hypermethylated cytosine nucleotides in the FRAXG CpG island will have an increased likelihood of developing short stature.

5. A primer set for amplifying a fragment of genomic DNA from a subject containing FRAXG, comprising:
a) a forward primer, identical to a contiguous sequence of nucleotides in that part of SEQ ID NO. 4 that is left of FRAXG; and
b) a reverse primer, complementary to a contiguous sequence of nucleotides in that part of SEQ ID NO. 4 that is right of FRAXG.

6. The primer set according to claim 5, wherein each primer has a length from about 10 to 30 nucleotides.

7. The primer set according to claim 6, wherein each primer has a length from about 15 to 25 nucleotides.

8. The primer set according to claim 7, wherein each primer has a length from about 18 to 22 nucleotides.

9. The primer set according to claim 5, wherein the G+C content of each primer is between 40% and 60%, and wherein the percentage of G+C content in the 3′ end of each primer is higher than the percentage of G+C content in the 5′ end of each primer.

10. A primer set according to claim 5, wherein the forward primer has the sequence set forth in SEQ ID NO:2 and the reverse primer has the sequence set forth in SEQ ID NO:3.

11. A primer set according to claim 5, wherein the forward primer has the sequence set forth in SEQ ID NO:10 and the reverse primer has the sequence set forth in SEQ ID NO:11.

12. A polynucleotide probe for determining the number of (CGG)ₙ/(CCG)ₙ nucleotide triplets within FRAXG, capable of hybridizing under stringent conditions to a region within SEQ ID NO. 4 (FIG. 14) that contains all or part of FRAXG.

13. The polynucleotide probe according to claim 12, wherein the probe has a length from about 14 to 80 nucleotides.

14. The polynucleotide probe according to claim 12, wherein the probe has a length from about 15 to 20 nucleotides.

15. The polynucleotide probe according to claim 12, wherein the probe comprises a sequence having multiple CGG repeats.

16. The polynucleotide probe according to claim 15, wherein the probe comprises a sequence having at least seven CGG repeats.

17. The polynucleotide probe according to claim 12, wherein the probe comprises all or a portion of the 770 bp HpaI-EcoRI fragment.

18. A kit for determining the number of (CGG)ₙ/(CCG)ₙ nucleotide triplets within FRAXG, comprising:
a) a primer set for amplifying a fragment of genomic DNA from a subject containing FRAXG, comprising a forward primer, identical to a contiguous sequence of nucleotides in that part of SEQ ID NO. 4 that is left of FRAXG, and a reverse primer, complementary to a contiguous sequence of nucleotides in that part of SEQ ID NO. 4 that is right of FRAXG; and
b) a polynucleotide probe for determining the number of (CGG)ₙ/(CCG)ₙ nucleotide triplets within FRAXG, capable of hybridizing under stringent conditions to a region within SEQ ID NO. 4 (FIG. 14) that contains all or part of FRAXG.

19. A primer set for amplifying a fragment of genomic DNA from a subject containing FRAXG, comprising:
a) a forward primer, identical to a contiguous sequence of nucleotides in that part of SEQ ID NO. 4 that is left of FRAXG; and
b) a reverse primer, complementary to a contiguous sequence of nucleotides in that part of SEQ ID NO. 4 that is right of FRAXG,
wherein at least one primer in the set has a sequence which is complimentary to a region of the FRAXG CpG island that contains CpG dinucleotides and within which a methylation-sensitive restriction endonuclease recognition site is present.

20. A cell line containing one or more FRAXG alleles that have a number of (CGG)ₙ/(CCG)ₙ nucleotide triplets that is significantly greater than the average number of triplets from a normal population of individuals.
